(19) United States
(12) Patent Application Publication
(10) Pub. No.: US 2016/0101204 A1
(43) Pub. Date: Apr. 14, 2016

(54) Soluble Fibrous Structures and Methods for Making Same

(71) Applicant: The Procter & Gamble Company, Cincinnati, OH (US)

(72) Inventors: Matthew Lawrence Lynch, Mariemont, OH (US); Brandon Philip Illie, Felicity, OH (US); Min Mao, Deerfield Township, OH (US); David Charles Oertel, Cincinnati, OH (US); Andreas Josef Dreher, Cincinnati, OH (US)

(73) Assignee: The Procter & Gamble Company

(21) Appl. No.: 14/879,131

(22) Filed: Oct. 9, 2015

Related U.S. Application Data
(60) Provisional application No. 62/062,185, filed on Oct. 10, 2014.

(51) Int. Cl. A61L 9/012; C11D 17/04; C11B 9/00

(52) U.S. Cl. CPC A61L 9/012; C11B 9/00; C11D 17/042

(57) ABSTRACT

Soluble fibrous structures and more particularly soluble fibrous structures that contain one or more fibrous elements, such as filaments, having one or more fibrous element-forming materials and one or more active agents present within the fibrous elements, wherein the fibrous structure exhibits improved dissolution properties compared to known soluble fibrous structures, and method for making such improved fibrous structures are provided.

### FIELD OF THE INVENTION

The present invention relates to soluble fibrous structures and more particularly to soluble fibrous structures that comprise one or more fibrous elements, such as filaments, comprising one or more fibrous element-forming materials and one or more active agents present within the fibrous elements, wherein the fibrous structure exhibits improved dissolution properties compared to known soluble fibrous structures, and method for making such improved fibrous structures while exhibiting consumer acceptable physical properties, such as strength, softness, elongation, and modulus.

### BACKGROUND OF THE INVENTION

Soluble fibrous structures comprising one or more fibrous elements, such as filaments, comprising one or more fibrous element-forming materials, such as a polymer, and one or more active agents present within the fibrous elements are known in the art. These known soluble fibrous structures typically comprise a plurality of filaments comprising fibrous element-forming materials, for example polar solvent-soluble polymers such as polyvinyl alcohol, and active agents, such as surfactants. Such known soluble fibrous structures may be used to deliver active agents, such as detergent compositions, in applications such as cleaning. In such cleaning applications, a desired amount of the soluble fibrous structure is placed in a liquid, such as water, the dissolution of the soluble fibrous structure and filaments is initiated thus releasing the active agents from the filaments. However, it is far too common that the soluble fibrous structures and filaments do not completely and/or satisfactorily dissolve under their conditions of intended use and result in an unsightly gel residue without completely delivering the intended benefit of the soluble fibrous structure.

As can be seen, dissolution of soluble fibrous structures is a key attribute and key consumer need. Accordingly, one problem with known soluble fibrous structures is that they fail to completely and/or satisfactorily dissolve under conditions of intended use, especially under consumer relevant times, thus failing to deliver, at least completely, their intended benefit. The problem is associated with how truly effectively the liquid, such as water, moves into and/or through the soluble fibrous structure and/or fibrous elements making up the soluble fibrous structure. Having one portion of a soluble fibrous structure and/or a few filaments dissolve quickly upon contacting water, but then halt and/or retard and/or inhibit the water flow into and/or through the remaining portion of the soluble fibrous structure such that dissolution of the remaining portion of the soluble fibrous structure is less than satisfactory to consumers and is thus not consumer acceptable.

Accordingly, there is a need for soluble fibrous structures that completely and/or satisfactorily dissolve under conditions of intended use, especially under consumer relevant times, to deliver their intended benefit without the negatives associated with known soluble fibrous structures. Also, there is a need for soluble fibrous structures that completely and/or satisfactorily dissolve under conditions of intended use while also exhibiting consumer acceptable strength, softness, elongation, and modulus.

### SUMMARY OF THE INVENTION

The present invention fulfills the needs described above by providing a soluble fibrous structure that completely and/or satisfactorily dissolves under conditions of intended use, especially under consumer relevant times, to deliver its intended benefit.

It has unexpectedly been found that the dissolution of soluble fibrous structures is influenced by the microstructure of the soluble fibrous structures, for example its propensity to wick the dissolving liquid, individual fibrous element hydration and/or swelling characteristics, and the viscosity of the dissolved soluble fibrous structure and/or fibrous elements making up the soluble fibrous structure, as well as the viscosity of the composition of the soluble fibrous structure and/or its fibrous elements such as filaments.

One solution to the problem identified above is to make a soluble fibrous structure having both a microstructure and composition such that the soluble fibrous structure exhibits improved dissolution. One way to achieve improved dissolution of the soluble fibrous structure is to have the soluble fibrous structure's combined microstructure and composition provide a desired soluble fibrous structure's Initial Water Propagation Rate as measured according to the Initial Water Propagation Rate Test Method described herein. It has unexpectedly been found that the soluble fibrous structures of the present invention exhibit an Initial Water Propagation Rate of greater than about 5.0×10⁻⁴ m/s as measured according to the Initial Water Propagation Rate Test Method described herein. The soluble fibrous structure's improved dissolution can be influenced by the soluble fibrous structure's fibrous element's Hydration Value as measured according to the Hydration Value Test Method described herein and/or Swelling Value as measured according to the Swelling Value Test Method described herein. It has surprisingly been found that the soluble fibrous structures of the present invention comprise one or more fibrous elements that exhibit a Hydration Value of greater than about 7.75×10⁻⁵ m/s^{1/2} as measured according to the Hydration Value Test Method described herein. It has also unexpectedly been found that the soluble fibrous structures of the present invention comprise one or more fibrous elements that exhibit a Swelling Value of less than about 2.05 as measured according to the Swelling Value Test Method described herein. Also, the soluble fibrous structure's improved dissolution can be influenced by the soluble fibrous structure's fibrous element's fibrous element-forming composition's Viscosity Value (pre-fibrous element formation and/or post-fibrous element formation, in other words, the Viscosity Value of the relevant fibrous element-forming composition, fibrous elements made therefrom, and soluble fibrous structure made therefrom) as measured according to the Viscosity Value Test Method described herein. It has surprisingly been found that the soluble fibrous structures of the present invention comprise fibrous elements comprising a fibrous element-forming composition and/or are made from a fibrous element-forming composition that exhibit a Viscosity Value of less than about 100 Pa·s as measured according to the Viscosity Value Test Method described herein.

The Initial Water Propagation Rate is set primarily by the fibrous structure composed from the fibrous elements. Not wishing to be bound by theory, it is believed that the Initial Water Propagation Rate is driven by capillary forces that draw water into the porous fibrous structure. The capillary forces are mostly governed by the characteristics of fibrous structure, which includes spacing between fibrous elements (e.g., pore size), density between fibrous elements (e.g. porosity), the size or effective diameter of the fibrous elements, the surface energy of the fibrous elements, surface texture of the fibrous elements, solid additives residing in the spacing and/or pores between fibrous elements. Fast Initial Water Propagation Rates (greater than about 5.0×10⁻⁴ m/s) are generally associated with fibrous structures which contain, for example, generally large capillary pressure (e.g. small contact angle and small spacing between the fibrous elements), large porosity (e.g. low density of fiber elements) and high permeability (e.g. large fiber radius). Unexpectedly, we have found that selection of the appropriate combinations of fibrous element-forming compositions, fibrous element characteristics, fibrous structure characteristics, and fibrous structure making processes, produce a soluble fibrous structure that contains the optimum combination of capillary pressure, porosity, and permeability, which yield an Initial Water Propagation Rate of greater than about 5.0×10⁻⁴ m/s as measured according to the Initial Water Propagation Rate Test Method described herein such that the soluble fibrous structure exhibits superior dissolution performance.

Hydration Value, not wishing to be bound by theory, indicates the rate at which fibrous elements uptake water and consequently the rate at which the fibrous elements expand in size. In other words, the Hydration Value addresses the question of how fast a fluid, for example water, penetrates into the fibrous elements causing them to expand. The expansion of the fibrous elements can further influence wetting and/or wicking rate wherein high Hydration Values may be associated with more rapid closing of pores in a fibrous structure thus one would expect high Hydration Values to inhibit and/or retard penetration of a fluid, such as water, into the fibrous structure. Hydration Values of greater than about 7.75×10⁻⁵ m/S^{1/2} as measured according to the Hydration Value Test Method described herein have unexpectedly been found to be sufficiently fast (high) to effectively minimize pore closure while maintaining effective fluid penetration and flow into the soluble fibrous structure and its fibrous elements of the present invention.

Swelling Value, not wishing to be bound by theory, indicates the degree to which the fibrous elements of a fibrous structure change in volume when hydrated. In other words, the Swelling Value addresses the question as to increase in volume per unit section of a fibrous element when hydrated completely. The volume growth of the fibrous element can further influence wetting and/or wicking rate wherein high Swelling Values (high swelling volume) can cause closing of pores in a fibrous structure thus inhibiting and/or retarding penetration of a fluid, such as water. Conversely, it is believed that low Swelling Values maintain and/or retard closing of the initial pores of the porous fibrous structure, thus maintaining the highest possible or superior fluid penetration and wicking rates for the fibrous structure. Surprisingly, it has been found that the fibrous element-forming compositions according to the present invention exemplified herein exhibit Swelling Values greater 0.5, but less than about 2.05 as measured according to the Swelling Value Test Method described herein. Swelling Values of less than about 2.05 as measured according to the Swelling Value Test Method described herein have unexpectedly been found to be sufficiently low to ensure effective fluid penetration and flow into the soluble fibrous structure and its fibrous elements of the present invention.

Viscosity, not wishing to be bound by theory, works in conjunction with the soluble fibrous structure and its fibrous element's fibrous element-forming composition by influencing the fluid propagation rate after the initial contact of the soluble fibrous structure with the fluid, such as water. It is believed that dissolution time of the soluble fibrous structure is reduced by ensuring fluid completely wicks into and wets the soluble fibrous structure prior to significant dissolution of the soluble fibrous structure's fibrous elements. The rate at which fluid propagates through the soluble fibrous structure is proportional not only to the capillary pressure described above but also inversely proportional to the viscosity of the fluid, such as water. Assuming this to be valid, then low viscous fluids generally move most rapidly through a soluble fibrous structure. Unexpectedly, we found that when the Viscosity Value of the soluble fibrous structure's fibrous element's fibrous element-forming composition (pre-fibrous element formation and/or post-fibrous element formation and/or post-soluble fibrous structure formation) is less than 100 Pa·s as measured according to the Viscosity Value Test Method described herein superior dissolution performance is achieved. Since viscosity and flow rate are inversely related, it is surprising that the Viscosity Value can be as high as 100 Pa·s while the soluble fibrous structure still maintains superior dissolution properties. Generally, the soluble fibrous structure's fibrous element's fibrous element-forming composition's Viscosity Values (pre-fibrous element formation and/or post-fibrous element formation and/or post-soluble fibrous structure formation) are achieved by adjusting the properties of the fibrous element-forming composition which then becomes the fibrous element formulation and ultimately the soluble fibrous structure's formulation. Viscosity of the fibrous element-forming composition can be reduced by (but not limited to) using low molecular weight polymers, inclusion of weak surfactants (do not form highly-viscous self-assembled structures during use), formulating with polymer blends, adjusting component levels such as the level of plasticizer, and a host of other formulation approaches.

In one example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure exhibits an Initial Water Propagation Rate of greater than about 5.0×10⁻⁴ m/s as measured according to the Initial Water Propagation Rate Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure comprises at least one fibrous element that exhibits a Hydration Value of greater than about 7.75×10⁻⁵ m/s^{1/2} as measured according to the Hydration Value Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure comprises at least one fibrous element that exhibits a Swelling Value of less than about 2.05 as measured according to the Swelling Value Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure comprises at least one fibrous element comprising a fibrous element-forming composition that exhibits a Viscosity Value of less than about 100 Pa·s as measured according to the Viscosity Value Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure comprises at least one fibrous element comprising a fibrous element-forming composition such that the fibrous element exhibits a Viscosity Value of less than about 100 Pa·s as measured according to the Viscosity Value Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure comprises at least one fibrous element comprising a fibrous element-forming composition such that the soluble fibrous structure exhibits a Viscosity Value of less than about 100 Pa·s as measured according to the Viscosity Value Test Method described herein, is provided.

In another example of the present invention, a soluble fibrous structure comprising a plurality of fibrous elements comprising one or more fibrous element-forming materials and one or more active agents that are releasable from the fibrous elements when exposed to conditions of intended use, wherein the soluble fibrous structure exhibits two or more and/or three or more, and/or four or more and/or all five of the following properties:

a. the soluble fibrous structure exhibits an Initial Water Propagation Rate of greater than about 5.0×10⁻⁴ m/s as measured according to the Initial Water Propagation Rate Test Method described herein;

b. at least one fibrous element within the soluble fibrous structure exhibits a Hydration Value of greater than about 7.75×10⁻⁵ m/s^{1/2} as measured according to the Hydration Value Test Method described herein;

c. at least one fibrous element within the soluble fibrous structure exhibits a Swelling Value of less than about 2.05 as measured according to the Swelling Value Test Method described herein;

d. at least one fibrous element within the soluble fibrous structure comprises a fibrous element-forming composition that exhibits a Viscosity Value of less than about 100 Pa·s as measured according to the Viscosity Value Test Method described herein;

e. at least one fibrous element within the soluble fibrous structure exhibits a Viscosity Value of less than about 100 Pa·s as measured according to the Viscosity Value Test Method described herein; and

f. the soluble fibrous structure exhibits a Viscosity Value of less than about 100 Pa·s as measured according to the Viscosity Value Test Method described herein, is provided.

In still another example of the present invention, a method for making a fibrous element-forming composition comprising the steps of:

a. providing one or more fibrous element-forming materials;

b. providing one or more active agents; and

c. mixing at least one fibrous element-forming material with at least one active agent to form a fibrous element-forming composition such that the fibrous element-forming composition exhibits a Viscosity Value of less than about 100 Pa·s as measured according to the Viscosity Value Test Method described herein, is provided.

In even another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition such that the fibrous element-forming composition exhibits a Viscosity Value of less than about 100 Pa·s as measured according to the Viscosity Value Test Method described herein; and

d. spinning the fibrous element-forming composition to produce one or more fibrous elements, is provided.

In even yet another example of the present invention, a method for making a soluble fibrous structure comprising the steps of:

a. providing one or more fibrous element-forming materials;

b. providing one or more active agents;

c. mixing at least one fibrous element-forming material with at least one active agent to form a fibrous element-forming composition;

d. spinning the fibrous element-forming composition to produce one or more fibrous elements; and

e. collecting the fibrous elements on a collection device, such as a belt, for example a patterned belt, such that a soluble fibrous structure that exhibits a Viscosity Value of less than about 100 Pa·s as measured according to the Viscosity Value Test Method described herein is formed, is provided.

In even yet another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition; and

d. spinning the fibrous element-forming composition to produce one or more fibrous elements such that at least one of the fibrous elements exhibits a Viscosity Value of less than about 100 Pa·s as measured according to the Viscosity Value Test Method described herein, is provided.

In even yet another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition;

d. spinning the fibrous element-forming composition to produce one or more fibrous elements; and

e. collecting the fibrous elements on a collection device, such as a belt, for example a patterned belt, such that a soluble fibrous structure that exhibits a Viscosity Value of less than about 100 Pa·s as measured according to the Viscosity Value Test Method described herein is formed, is provided.

In even yet another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition; and

d. spinning the fibrous element-forming composition to produce one or more fibrous elements such that at least one of the fibrous elements exhibits a Swelling Value of less than about 2.05 as measured according to the Swelling Value Test Method described herein, is provided.

In still even yet another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition; and

d. spinning the fibrous element-forming composition to produce one or more fibrous elements such that at least one of the fibrous elements exhibits a Hydration Value of greater than about 7.75×10⁻⁵ m/s^{1/2} as measured according to the Hydration Value Test Method described herein, is provided.

In even still another example of the present invention, a method for making a fibrous structure comprising the steps of:

a. providing one or more fibrous element-forming materials

b. providing one or more active agents;

c. mixing at least one fibrous element-forming material with at least one active agent to form a fibrous element-forming composition;

d. spinning the fibrous element-forming composition to produce a plurality of fibrous elements; and

e. collecting the plurality of fibrous elements on a collection device to form a fibrous structure such that at least one of the fibrous elements within the fibrous structure exhibits a Swelling Value of less than about 2.05 as measured according to the Swelling Value Test Method described herein, is provided.

In even still another example of the present invention, a method for making a fibrous structure comprising the steps of:

a. providing one or more fibrous element-forming materials

b. providing one or more active agents;

c. mixing at least one fibrous element-forming material with at least one active agent to form a fibrous element-forming composition;

d. spinning the fibrous element-forming composition to produce a plurality of fibrous elements; and

e. collecting the plurality of fibrous elements on a collection device to form a fibrous structure such that at least one of the fibrous elements of the fibrous structure exhibits a Hydration Value of greater than about 7.75×10⁻⁵ m/s^{1/2} as measured according to the Hydration Value Test Method described herein, is provided.

In even still another example of the present invention, a method for making a fibrous structure comprising the steps of:

a. providing one or more fibrous element-forming materials

b. providing one or more active agents;

c. mixing at least one fibrous element-forming material with at least one active agent to form a fibrous element-forming composition;

d. spinning the fibrous element-forming composition to produce a plurality of fibrous elements; and

e. collecting the plurality of fibrous elements on a collection device to form a fibrous structure such that the fibrous structure exhibits an Initial Water Propagation Rate of greater than about 5.0×10⁻⁴ m/s as measured according to the Initial Water Propagation Rate Test Method described herein, is provided.

In even yet another example of the present invention, a method for making a fibrous element comprising the steps of:

a. providing one or more fibrous element-forming materials;

b. providing one or more active agents;

c. mixing at least one fibrous element-forming material with at least one active agent to form a fibrous element-forming composition; and

d. spinning the fibrous element-forming composition to produce one or more fibrous elements such that at least one of the fibrous elements exhibits two or more of the following properties:
i. a Swelling Value of less than about 2.05 as measured according to the Swelling Value Test Method described herein;
ii. a Hydration Value of greater than about 7.75×10⁻⁵ m/s^{1/2} as measured according to the Hydration Value Test Method described herein; and
iii. a Viscosity Value of less than about 100 Pa·s as measured according to the Viscosity Value Test Method described herein, is provided.

In even yet another example of the present invention, a method for making a soluble fibrous structure comprising the steps of:

a. providing one or more fibrous element-forming materials;

b. providing one or more active agents;

c. mixing at least one fibrous element-forming material with at least one active agent to form a fibrous element-forming composition;

d. spinning the fibrous element-forming composition to produce one or more fibrous elements; and

e. collecting the fibrous elements on a collection device, such as a belt, for example a patterned belt, such that a soluble fibrous structure that exhibits the following properties:
i. an Initial Water Propagation Rate of greater than about 5.0×10⁻⁴ m/s as measured according to the Initial Water Propagation Rate Test Method described herein; and
ii. a Viscosity Value of less than about 100 Pa·s as measured according to the Viscosity Value Test Method described herein is formed, is provided.

Accordingly, the present invention provide novel soluble fibrous structures that exhibit improved dissolution properties compared to known soluble fibrous structures and methods for making same.

### BRIEF DESCRIPTION OF THE DRAWINGS

FIG. 1 is a schematic representation of an example of a fibrous element according to the present invention;

FIG. 2 is a schematic representation of an example of a soluble fibrous structure according to the present invention;

FIG. 3 is a schematic representation of an example of a process for making fibrous elements of the present invention;

FIG. 4 is a schematic representation of an example of a die with a magnified view used in the process of FIG. 3;

FIG. 5 is a front view of an example of a setup of equipment used in measuring dissolution according to the present invention;

FIG. 6 is a side view of FIG. 5; and

FIG. 7 is a partial top view of FIG. 6.

### DETAILED DESCRIPTION OF THE INVENTION

### Definitions

“Fibrous structure” as used herein means a structure that comprises one or more fibrous elements. In one example, a fibrous structure according to the present invention means an association of fibrous elements and particles that together form a structure, such as a unitary structure, capable of performing a function.

The fibrous structures of the present invention may be homogeneous or may be layered. If layered, the fibrous structures may comprise at least two and/or at least three and/or at least four and/or at least five layers, for example one or more fibrous element layers, one or more particle layers and/or one or more fibrous element/particle mixture layers. In one example, in a multiple layer fibrous structure, one or more layers may be formed and/or deposited directly upon an existing layer to form a fibrous structure whereas in a multi-ply fibrous structure, one or more existing fibrous structure plies may be combined, for example via thermal bonding, gluing, embossing, rodding, rotary knife aperturing, needlepunching, knurling, tufting, and/or other mechanical combining process, with one or more other existing fibrous structure plies to form the multi-ply fibrous structure.

In one example, the fibrous structure is a multi-ply fibrous structure that exhibits a basis weight of less than 10000 g/m² as measured according to the Basis Weight Test Method described herein.

In one example, the fibrous structure is a sheet of fibrous elements (fibers and/or filaments, such as continuous filaments), of any nature or origin, that have been formed into a web by any means, and may be bonded together by any means, with the exception of weaving or knitting. Felts obtained by wet milling are not soluble fibrous structures. In one example, a fibrous structure according to the present invention means an orderly arrangement of filaments within a structure in order to perform a function. In another example, a fibrous structure of the present invention is an arrangement comprising a plurality of two or more and/or three or more fibrous elements that are inter-entangled or otherwise associated with one another to form a fibrous structure. In yet another example, the fibrous structure of the present invention may comprise, in addition to the fibrous elements of the present invention, one or more solid additives, such as particulates and/or fibers.

In one example, the fibrous structure of the present invention is a “unitary fibrous structure.”

“Unitary fibrous structure” as used herein is an arrangement comprising a plurality of two or more and/or three or more fibrous elements that are inter-entangled or otherwise associated with one another to form a fibrous structure. A unitary fibrous structure of the present invention may be one or more plies within a multi-ply fibrous structure. In one example, a unitary fibrous structure of the present invention may comprise three or more different fibrous elements. In another example, a unitary fibrous structure of the present invention may comprise two different fibrous elements, for example a co-formed fibrous structure, upon which a different fibrous elements are deposited to form a fibrous structure comprising three or more different fibrous elements. In one example, a fibrous structure may comprise soluble, for example water-soluble, fibrous elements and insoluble, for example water insoluble fibrous elements.

“Soluble fibrous structure” as used herein means the fibrous structure and/or components thereof, for example greater than 0.5% and/or greater than 1% and/or greater than 5% and/or greater than 10% and/or greater than 25% and/or greater than 50% and/or greater than 75% and/or greater than 90% and/or greater than 95% and/or about 100% by weight of the fibrous structure is soluble, for example polar solvent-soluble such as water-soluble. In one example, the soluble fibrous structure comprises fibrous elements wherein at least 50% and/or greater than 75% and/or greater than 90% and/or greater than 95% and/or about 100% by weight of the fibrous elements within the soluble fibrous structure are soluble.

The soluble fibrous structure comprises a plurality of fibrous elements. In one example, the soluble fibrous structure comprises two or more and/or three or more different fibrous elements.

The soluble fibrous structure and/or fibrous elements thereof, for example filaments, making up the soluble fibrous structure may comprise one or more active agents, for example a fabric care active agent, a dishwashing active agent, a hard surface active agent, a hair care active agent, a floor care active agent, a skin care active agent, an oral care active agent, a medicinal active agent, carpet care active agents, surface care active agents, air care active agents, and mixtures thereof. In one example, a soluble fibrous structure and/or fibrous elements thereof of the present invention comprises one or more surfactants, one or more enzymes (such as in the form of an enzyme prill), one or more perfumes and/or one or more suds suppressors. In another example, a soluble fibrous structure and/or fibrous elements thereof of the present invention comprises a builder and/or a chelating agent. In another example, a soluble fibrous structure and/or fibrous elements thereof of the present invention comprises a bleaching agent (such as an encapsulated bleaching agent). In still another example, a soluble fibrous structure and/or fibrous elements thereof of the present invention comprises one or more surfactants and optionally, one or more perfumes.

In one example, the soluble fibrous structure of the present invention is a water-soluble fibrous structure.

In one example, the soluble fibrous structure of the present invention exhibits a basis weight of less than 10000 g/m² and/or less than 5000 g/m² and/or less than 4000 g/m² and/or less than 2000 g/m² and/or less than 1000 g/m² and/or less than 500 g/m² as measured according to the Basis Weight Test Method described herein.

“Fibrous element” as used herein means an elongate particulate having a length greatly exceeding its average diameter, i.e. a length to average diameter ratio of at least about 10. A fibrous element may be a filament or a fiber. In one example, the fibrous element is a single fibrous element or a yarn comprising a plurality of fibrous elements. In another example, the fibrous element is a single fibrous element.

The fibrous elements of the present invention may be spun from a fibrous element-forming compositions also referred to as fibrous element-forming compositions via suitable spinning process operations, such as meltblowing, spunbonding, electro-spinning, and/or rotary spinning.

The fibrous elements of the present invention may be monocomponent and/or multicomponent. For example, the fibrous elements may comprise bicomponent fibers and/or filaments. The bicomponent fibers and/or filaments may be in any form, such as side-by-side, core and sheath, islands-in-the-sea and the like.

In one example, the fibrous element, which may be a filament and/or a fiber and/or a filament that has been cut to smaller fragments (fibers) of the filament may exhibit a length of greater than or equal to 0.254 cm (0.1 in.) and/or greater than or equal to 1.27 cm (0.5 in.) and/or greater than or equal to 2.54 cm (1.0 in.) and/or greater than or equal to 5.08 cm (2 in.) and/or greater than or equal to 7.62 cm (3 in.) and/or greater than or equal to 10.16 cm (4 in.) and/or greater than or equal to 15.24 cm (6 in.). In one example, a fiber of the present invention exhibits a length of less than 5.08 cm (2 in.).

“Filament” as used herein means an elongate particulate as described above. In one example, a filament exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers. Filaments are relatively longer than fibers. Non-limiting examples of filaments include meltblown and/or spunbond filaments.

In one example, one or more fibers may be formed from a filament of the present invention, such as when the filaments are cut to shorter lengths. Thus, in one example, the present invention also includes a fiber made from a filament of the present invention, such as a fiber comprising one or more fibrous element-forming materials and one or more additives, such as active agents. Therefore, references to filament and/or filaments of the present invention herein also include fibers made from such filament and/or filaments unless otherwise noted. Fibers are typically considered discontinuous in nature relative to filaments, which are considered continuous in nature.

Non-limiting examples of fibrous elements include meltblown and/or spunbond fibrous elements. Non-limiting examples of polymers that can be spun into fibrous elements include natural polymers, such as starch, starch derivatives, cellulose, such as rayon and/or lyocell, and cellulose derivatives, hemicellulose, hemicellulose derivatives, and synthetic polymers including, but not limited to thermoplastic polymer fibrous elements, such as polyesters, nylons, polyolefins such as polypropylene filaments, polyethylene filaments, and biodegradable thermoplastic fibers such as polylactic acid filaments, polyhydroxyalkanoate filaments, polyesteramide filaments and polycaprolactone filaments. Depending upon the polymer and/or composition from which the fibrous elements are made, the fibrous elements may be soluble or insoluble.

“Fibrous element-forming composition” as used herein means a composition that is suitable for making a fibrous element, for example a filament, of the present invention such as by meltblowing and/or spunbonding. The fibrous element-forming composition comprises one or more fibrous element-forming materials that exhibit properties that make them suitable for spinning into a fibrous element, for example a filament. In one example, the fibrous element-forming material comprises a polymer. In addition to one or more fibrous element-forming materials, the fibrous element-forming composition may comprise one or more additives, for example one or more active agents. In addition, the fibrous element-forming composition may comprise one or more polar solvents, such as water, into which one or more, for example all, of the fibrous element-forming materials and/or one or more, for example all, of the active agents are dissolved and/or dispersed.

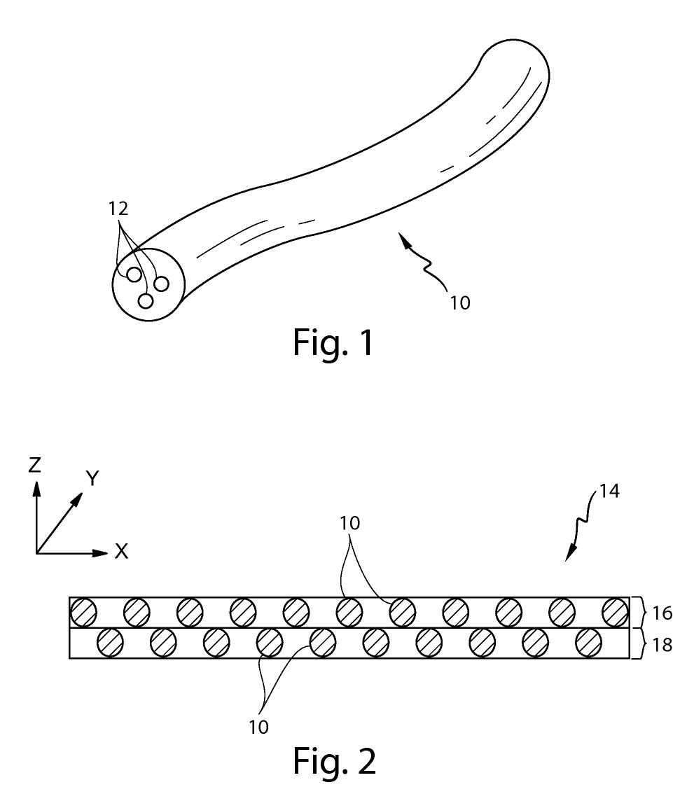

In one example as shown in FIG. 1 a fibrous element 10, for example a filament, of the present invention made from a fibrous element-forming composition of the present invention is such that one or more additives, for example one or more active agents 12, may be present in the fibrous element 10, for example filament, rather than on the fibrous element 10, such as a coating composition. The total level of fibrous element-forming materials and total level of active agents present in the fibrous element-forming composition may be any suitable amount so long as the fibrous elements, for example filaments, of the present invention are produced therefrom.

In one example, one or more additives, such as active agents, may be present in the fibrous element and one or more additional additives, such as active agents, may be present on a surface of the fibrous element. In another example, a fibrous element of the present invention may comprise one or more additives, such as active agents, that are present in the fibrous element when originally made, but then bloom to a surface of the fibrous element prior to and/or when exposed to conditions of intended use of the fibrous element.

“Fibrous element-forming material” as used herein means a material, such as a polymer or monomers capable of producing a polymer that exhibits properties suitable for making a fibrous element. In one example, the fibrous element-forming material comprises one or more substituted polymers such as an anionic, cationic, zwitterionic, and/or nonionic polymer. In another example, the polymer may comprise a hydroxyl polymer, such as a polyvinyl alcohol (“PVOH”) and/or a polysaccharide, such as starch and/or a starch derivative, such as an ethoxylated starch and/or acid-thinned starch. In another example, the polymer may comprise polyethylenes and/or terephthalates. In yet another example, the fibrous element-forming material is a polar solvent-soluble material.

“Particle” as used herein means a solid additive, such as a powder, granule, encapsulate, microcapsule, such as a perfume microcapsule, and/or prill. In one example, the fibrous elements and/or fibrous structures of the present invention may comprise one or more particles. The particles may be intra-fibrous element (within the fibrous elements, like the active agents) and/or inter-fibrous element (between fibrous elements within a soluble fibrous structure. Non-limiting examples of fibrous elements and/or fibrous structures comprising particles are described in US 2013/0172226 which is incorporated herein by reference. In one example, the particle exhibits a median particle size of 1600 μm or less as measured according to the Median Particle Size Test Method described herein. In another example, the particle exhibits a median particle size of from about 1 μm to about 1600 μm and/or from about 1 μm to about 800 μm and/or from about 5 μm to about 500 μm and/or from about 10 μm to about 300 μm and/or from about 10 μm to about 100 μm and/or from about 10 μm to about 50 μm and/or from about 10 μm to about 30 μm as measured according to the Median Particle Size Test Method described herein. The shape of the particle can be in the form of spheres, rods, plates, tubes, squares, rectangles, discs, stars, fibers or have regular or irregular random forms.

“Active agent-containing particle” as used herein means a solid additive comprising one or more active agents. In one example, the active agent-containing particle is an active agent in the form of a particle (in other words, the particle comprises 100% active agent(s)). The active agent-containing particle may exhibit a median particle size of 1600 μm or less as measured according to the Median Particle Size Test Method described herein. In another example, the active agent-containing particle exhibits a median particle size of from about 1 μm to about 1600 μm and/or from about 1 μm to about 800 μm and/or from about 5 μm to about 500 μm and/or from about 10 μm to about 300 μm and/or from about 10 μm to about 100 μm and/or from about 10 μm to about 50 μm and/or from about 10 μm to about 30 μm as measured according to the Median Particle Size Test Method described herein. In one example, one or more of the active agents is in the form of a particle that exhibits a median particle size of 20 μm or less as measured according to the Median Particle Size Test Method described herein.

In one example of the present invention, the fibrous structure comprises a plurality of particles, for example active agent-containing particles, and a plurality of fibrous elements in a weight ratio of particles, for example active agent-containing particles, to fibrous elements of 1:100 or greater and/or 1:50 or greater and/or 1:10 or greater and/or 1:3 or greater and/or 1:2 or greater and/or 1:1 or greater and/or from about 7:1 to about 1:100 and/or from about 7:1 to about 1:50 and/or from about 7:1 to about 1:10 and/or from about 7:1 to about 1:3 and/or from about 6:1 to 1:2 and/or from about 5:1 to about 1:1 and/or from about 4:1 to about 1:1 and/or from about 3:1 to about 1.5:1.

In another example of the present invention, the fibrous structure comprises a plurality of particles, for example active agent-containing particles, and a plurality of fibrous elements in a weight ratio of particles, for example active agent-containing particles, to fibrous elements of from about 7:1 to about 1:1 and/or from about 7:1 to about 1.5:1 and/or from about 7:1 to about 3:1 and/or from about 6:1 to about 3:1.

In yet another example of the present invention, the fibrous structure comprises a plurality of particles, for example active agent-containing particles, and a plurality of fibrous elements in a weight ratio of particles, for example active agent-containing particles, to fibrous elements of from about 1:1 to about 1:100 and/or from about 1:2 to about 1:50 and/or from about 1:3 to about 1:50 and/or from about 1:3 to about 1:10.

In another example, the fibrous structure of the present invention comprises a plurality of particles, for example active agent-containing particles, at a particle basis weight of greater than 1 g/m² and/or greater than 10 g/m² and/or greater than 20 g/m² and/or greater than 30 g/m² and/or greater than 40 g/m² and/or from about 1 g/m² to about 5000 g/m² and/or to about 3500 g/m² and/or to about 2000 g/m² and/or from about 1 g/m² to about 1000 g/m² and/or from about 10 g/m² to about 400 g/m² and/or from about 20 g/m² to about 300 g/m² and/or from about 30 g/m² to about 200 g/m² and/or from about 40 g/m² to about 100 g/m² as measured by the Basis Weight Test Method described herein.

In another example, the fibrous structure of the present invention comprises a plurality of fibrous elements at a basis weight of greater than 1 g/m² and/or greater than 10 g/m² and/or greater than 20 g/m² and/or greater than 30 g/m² and/or greater than 40 g/m² and/or from about 1 g/m² to about 10000 g/m² and/or from about 10 g/m² to about 5000 g/m² and/or to about 3000 g/m² and/or to about 2000 g/m² and/or from about 20 g/m² to about 2000 g/m² and/or from about 30 g/m² to about 1000 g/m² and/or from about 30 g/m² to about 500 g/m² and/or from about 30 g/m² to about 300 g/m² and/or from about 40 g/m² to about 100 g/m² and/or from about 40 g/m² to about 80 g/m² as measured by the Basis Weight Test Method described herein. In one example, the fibrous structure comprises two or more layers wherein fibrous elements are present in at least one of the layers at a basis weight of from about 1 g/m² to about 500 g/m².

“Additive” as used herein means any material present in the fibrous element of the present invention that is not a fibrous element-forming material. In one example, an additive comprises an active agent. In another example, an additive comprises a processing aid. In still another example, an additive comprises a filler. In one example, an additive comprises any material present in the fibrous element that its absence from the fibrous element would not result in the fibrous element losing its fibrous element structure, in other words, its absence does not result in the fibrous element losing its solid form. In another example, an additive, for example an active agent, comprises a non-polymer material.

In another example, an additive comprises a plasticizer for the fibrous element. Non-limiting examples of suitable plasticizers for the present invention include polyols, copolyols, polycarboxylic acids, polyesters and dimethicone copolyols. Examples of useful polyols include, but are not limited to, glycerin, diglycerin, propylene glycol, ethylene glycol, butylene glycol, pentylene glycol, cyclohexane dimethanol, hexanediol, 2,2,4-trimethylpentane-1,3-diol, polyethylene glycol (200-600), pentaerythritol, sugar alcohols such as sorbitol, manitol, lactitol and other mono- and polyhydric low molecular weight alcohols (e.g., C2-C8 alcohols); mono di- and oligo-saccharides such as fructose, glucose, sucrose, maltose, lactose, high fructose corn syrup solids, and dextrins, and ascorbic acid.

In one example, the plasticizer includes glycerin and/or propylene glycol and/or glycerol derivatives such as propoxylated glycerol. In still another example, the plasticizer is selected from the group consisting of glycerin, ethylene glycol, polyethylene glycol, propylene glycol, glycidol, urea, sorbitol, xylitol, maltitol, sugars, ethylene bisformamide, amino acids, and mixtures thereof

In another example, an additive comprises a crosslinking agent suitable for crosslinking one or more of the fibrous element-forming materials present in the fibrous elements of the present invention. In one example, the crosslinking agent comprises a crosslinking agent capable of crosslinking hydroxyl polymers together, for example via the hydroxyl polymers hydroxyl moieties. Non-limiting examples of suitable crosslinking agents include imidazolidinones, polycarboxylic acids and mixtures thereof. In one example, the crosslinking agent comprises a urea glyoxal adduct crosslinking agent, for example a dihydroxyimidazolidinone, such as dihydroxyethylene urea (“DHEU”). A crosslinking agent can be present in the fibrous element-forming composition and/or fibrous element of the present invention to control the fibrous element's solubility and/or dissolution in a solvent, such as a polar solvent.

In another example, an additive comprises a rheology modifier, such as a shear modifier and/or an extensional modifier. Non-limiting examples of rheology modifiers include but not limited to polyacrylamide, polyurethanes and polyacrylates that may be used in the fibrous elements of the present invention. Non-limiting examples of rheology modifiers are commercially available from The Dow Chemical Company (Midland, Mich.).

In yet another example, an additive comprises one or more colors and/or dyes that are incorporated into the fibrous elements of the present invention to provide a visual signal when the fibrous elements are exposed to conditions of intended use and/or when an active agent is released from the fibrous elements and/or when the fibrous element's morphology changes.

In still yet another example, an additive comprises one or more release agents and/or lubricants. Non-limiting examples of suitable release agents and/or lubricants include fatty acids, fatty acid salts, fatty alcohols, fatty esters, sulfonated fatty acid esters, fatty amine acetates, fatty amide, silicones, aminosilicones, fluoropolymers, and mixtures thereof. In one example, the release agents and/or lubricants are applied to the fibrous element, in other words, after the fibrous element is formed. In one example, one or more release agents/lubricants are applied to the fibrous element prior to collecting the fibrous elements on a collection device to form a soluble fibrous structure. In another example, one or more release agents/lubricants are applied to a soluble fibrous structure formed from the fibrous elements of the present invention prior to contacting one or more soluble fibrous structures, such as in a stack of soluble fibrous structures. In yet another example, one or more release agents/lubricants are applied to the fibrous element of the present invention and/or soluble fibrous structure comprising the fibrous element prior to the fibrous element and/or soluble fibrous structure contacting a surface, such as a surface of equipment used in a processing system so as to facilitate removal of the fibrous element and/or soluble fibrous structure and/or to avoid layers of fibrous elements and/or soluble fibrous structures of the present invention sticking to one another, even inadvertently. In one example, the release agents/lubricants comprise particulates.

In even still yet another example, an additive comprises one or more anti-blocking and/or detackifying agents. Non-limiting examples of suitable anti-blocking and/or detackifying agents include starches, starch derivatives, crosslinked polyvinylpyrrolidone, crosslinked cellulose, microcrystalline cellulose, silica, metallic oxides, calcium carbonate, talc, mica, and mixtures thereof.

“Conditions of intended use” as used herein means the temperature, physical, chemical, and/or mechanical conditions that a fibrous element of the present invention is exposed to when the fibrous element is used for one or more of its designed purposes. For example, if a fibrous element and/or a soluble fibrous structure comprising a fibrous element are designed to be used in a washing machine for laundry care purposes, the conditions of intended use will include that temperature, chemical, physical and/or mechanical conditions present in a washing machine, including any wash water, during a laundry washing operation. In another example, if a fibrous element and/or a soluble fibrous structure comprising a fibrous element are designed to be used by a human as a shampoo for hair care purposes, the conditions of intended use will include that temperature, chemical, physical and/or mechanical conditions present during the shampooing of the human's hair. Likewise, if a fibrous element and/or soluble fibrous structure comprising a fibrous element is designed to be used in a dishwashing operation, by hand or by a dishwashing machine, the conditions of intended use will include the temperature, chemical, physical and/or mechanical conditions present in a dishwashing water and/or dishwashing machine, during the dishwashing operation.

“Active agent” as used herein means an additive that produces an intended effect in an environment external to a fibrous element and/or soluble fibrous structure comprising the fibrous element of the present, such as when the fibrous element is exposed to conditions of intended use of the fibrous element and/or soluble fibrous structure comprising the fibrous element. In one example, an active agent comprises an additive that treats a surface, such as a hard surface (i.e., kitchen countertops, bath tubs, toilets, toilet bowls, sinks, floors, walls, teeth, cars, windows, mirrors, dishes) and/or a soft surface (i.e., fabric, hair, skin, carpet, crops, plants). In another example, an active agent comprises an additive that creates a chemical reaction (i.e., foaming, fizzing, coloring, warming, cooling, lathering, disinfecting and/or clarifying and/or chlorinating, such as in clarifying water and/or disinfecting water and/or chlorinating water). In yet another example, an active agent comprises an additive that treats an environment (i.e., deodorizes, purifies, perfumes air). In one example, the active agent is formed in situ, such as during the formation of the fibrous element containing the active agent, for example the fibrous element may comprise a water-soluble polymer (e.g., starch) and a surfactant (e.g., anionic surfactant), which may create a polymer complex or coacervate that functions as the active agent used to treat fabric surfaces.

“Treats” as used herein with respect to treating a surface means that the active agent provides a benefit to a surface or environment. Treats includes regulating and/or immediately improving a surface's or environment's appearance, cleanliness, smell, purity and/or feel. In one example treating in reference to treating a keratinous tissue (for example skin and/or hair) surface means regulating and/or immediately improving the keratinous tissue's cosmetic appearance and/or feel. For instance, “regulating skin, hair, or nail (keratinous tissue) condition” includes: thickening of skin, hair, or nails (e.g., building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

In another example, treating means removing stains and/or odors from fabric articles, such as clothes, towels, linens, and/or hard surfaces, such as countertops and/or dishware including pots and pans.

“Fabric care active agent” as used herein means an active agent that when applied to fabric provides a benefit and/or improvement to the fabric. Non-limiting examples of benefits and/or improvements to fabric include cleaning (for example by surfactants), stain removal, stain reduction, wrinkle removal, color restoration, static control, wrinkle resistance, permanent press, wear reduction, wear resistance, pill removal, pill resistance, soil removal, soil resistance (including soil release), shape retention, shrinkage reduction, softness, fragrance, anti-bacterial, anti-viral, odor resistance, and odor removal.

“Dishwashing active agent” as used herein means an active agent that when applied to dishware, glassware, pots, pans, utensils, and/or cooking sheets provides a benefit and/or improvement to the dishware, glassware, plastic items, pots, pans and/or cooking sheets. Non-limiting example of benefits and/or improvements to the dishware, glassware, plastic items, pots, pans, utensils, and/or cooking sheets include food and/or soil removal, cleaning (for example by surfactants) stain removal, stain reduction, grease removal, water spot removal and/or water spot prevention, glass and metal care, sanitization, shining, and polishing.

“Hard surface active agent” as used herein means an active agent when applied to floors, countertops, sinks, windows, mirrors, showers, baths, and/or toilets provides a benefit and/or improvement to the floors, countertops, sinks, windows, mirrors, showers, baths, and/or toilets. Non-limiting example of benefits and/or improvements to the floors, countertops, sinks, windows, mirrors, showers, baths, and/or toilets include food and/or soil removal, cleaning (for example by surfactants), stain removal, stain reduction, grease removal, water spot removal and/or water spot prevention, limescale removal, disinfection, shining, polishing, and freshening.

“Beauty benefit active agent,” as used herein, refers to an active agent that can deliver one or more beauty benefits.

“Skin care active agent” as used herein, means an active agent that when applied to the skin provides a benefit or improvement to the skin. It is to be understood that skin care active agents are useful not only for application to skin, but also to hair, scalp, nails and other mammalian keratinous tissue.

“Hair care active agent” as used herein, means an active agent that when applied to mammalian hair provides a benefit and/or improvement to the hair. Non-limiting examples of benefits and/or improvements to hair include softness, static control, hair repair, dandruff removal, dandruff resistance, hair coloring, shape retention, hair retention, and hair growth.

“Weight ratio” as used herein means the dry fibrous element, for example filament, basis and/or dry fibrous element-forming material (g or %) on a dry weight basis in the fibrous element, for example filament, to the weight of additive, such as active agent(s) (g or %) on a dry weight basis in the fibrous element, for example filament.

“Hydroxyl polymer” as used herein includes any hydroxyl-containing polymer that can be incorporated into a fibrous element of the present invention, for example as a fibrous element-forming material. In one example, the hydroxyl polymer of the present invention includes greater than 10% and/or greater than 20% and/or greater than 25% by weight hydroxyl moieties.

“Biodegradable” as used herein means, with respect to a material, such as a fibrous element as a whole and/or a polymer within a fibrous element, such as a fibrous element-forming material, that the fibrous element and/or polymer is capable of undergoing and/or does undergo physical, chemical, thermal and/or biological degradation in a municipal solid waste composting facility such that at least 5% and/or at least 7% and/or at least 10% of the original fibrous element and/or polymer is converted into carbon dioxide after 30 days as measured according to the OECD (1992) Guideline for the Testing of Chemicals 301B; Ready Biodegradability—CO₂ Evolution (Modified Sturm Test) Test incorporated herein by reference.

“Non-biodegradable” as used herein means, with respect to a material, such as a fibrous element as a whole and/or a polymer within a fibrous element, such as a fibrous element-forming material, that the fibrous element and/or polymer is not capable of undergoing physical, chemical, thermal and/or biological degradation in a municipal solid waste composting facility such that at least 5% of the original fibrous element and/or polymer is converted into carbon dioxide after 30 days as measured according to the OECD (1992) Guideline for the Testing of Chemicals 301B; Ready Biodegradability—CO₂ Evolution (Modified Sturm Test) Test incorporated herein by reference.

“Non-thermoplastic” as used herein means, with respect to a material, such as a fibrous element as a whole and/or a polymer within a fibrous element, such as a fibrous element-forming material, that the fibrous element and/or polymer exhibits no melting point and/or softening point, which allows it to flow under pressure, in the absence of a plasticizer, such as water, glycerin, sorbitol, urea and the like.

“Non-thermoplastic, biodegradable fibrous element” as used herein means a fibrous element that exhibits the properties of being biodegradable and non-thermoplastic as defined above.

“Non-thermoplastic, non-biodegradable fibrous element” as used herein means a fibrous element that exhibits the properties of being non-biodegradable and non-thermoplastic as defined above.

“Thermoplastic” as used herein means, with respect to a material, such as a fibrous element as a whole and/or a polymer within a fibrous element, such as a fibrous element-forming material, that the fibrous element and/or polymer exhibits a melting point and/or softening point at a certain temperature, which allows it to flow under pressure, in the absence of a plasticizer

“Thermoplastic, biodegradable fibrous element” as used herein means a fibrous element that exhibits the properties of being biodegradable and thermoplastic as defined above.

“Thermoplastic, non-biodegradable fibrous element” as used herein means a fibrous element that exhibits the properties of being non-biodegradable and thermoplastic as defined above.

“Non-cellulose-containing” as used herein means that less than 5% and/or less than 3% and/or less than 1% and/or less than 0.1% and/or 0% by weight of cellulose polymer, cellulose derivative polymer and/or cellulose copolymer is present in fibrous element. In one example, “non-cellulose-containing” means that less than 5% and/or less than 3% and/or less than 1% and/or less than 0.1% and/or 0% by weight of cellulose polymer is present in fibrous element.

“Polar solvent-soluble material” as used herein means a material that is miscible in a polar solvent. In one example, a polar solvent-soluble material is miscible in alcohol and/or water. In other words, a polar solvent-soluble material is a material that is capable of forming a stable (does not phase separate for greater than 5 minutes after forming the homogeneous solution) homogeneous solution with a polar solvent, such as alcohol and/or water at ambient conditions.

“Alcohol-soluble material” as used herein means a material that is miscible in alcohol. In other words, a material that is capable of forming a stable (does not phase separate for greater than 5 minutes after forming the homogeneous solution) homogeneous solution with an alcohol at ambient conditions.

“Water-soluble material” as used herein means a material that is miscible in water. In other words, a material that is capable of forming a stable (does not separate for greater than 5 minutes after forming the homogeneous solution) homogeneous solution with water at ambient conditions.

“Non-polar solvent-soluble material” as used herein means a material that is miscible in a non-polar solvent. In other words, a non-polar solvent-soluble material is a material that is capable of forming a stable (does not phase separate for greater than 5 minutes after forming the homogeneous solution) homogeneous solution with a non-polar solvent.

“Ambient conditions” as used herein means 73° F.±4° F. (about 23° C.±2.2° C.) and a relative humidity of 50%±10%.

“Weight average molecular weight” as used herein means the weight average molecular weight as determined using the Weight Average Molecular Weight Test Method described herein.

“Length” as used herein, with respect to a fibrous element, means the length along the longest axis of the fibrous element from one terminus to the other terminus. If a fibrous element has a kink, curl or curves in it, then the length is the length along the entire path of the fibrous element.

“Diameter” as used herein, with respect to a fibrous element, is measured according to the Diameter Test Method described herein. In one example, a fibrous element of the present invention exhibits a diameter of less than 100 μm and/or less than 75 μm and/or less than 50 μm and/or less than 25 μm and/or less than 20 μm and/or less than 15 μm and/or less than 10 μm and/or less than 6 μm and/or greater than 1 μm and/or greater than 3 μm.

“Triggering condition” as used herein in one example means anything, as an act or event, that serves as a stimulus and initiates or precipitates a change in the fibrous element, such as a loss or altering of the fibrous element's physical structure and/or a release of an additive, such as an active agent. In another example, the triggering condition may be present in an environment, such as water, when a fibrous element and/or soluble fibrous structure and/or film of the present invention are added to the water. In other words, nothing changes in the water except for the fact that the fibrous element and/or soluble fibrous structure and/or film of the present invention are added to the water.

“Morphology changes” as used herein with respect to a fibrous element's morphology changing means that the fibrous element experiences a change in its physical structure. Non-limiting examples of morphology changes for a fibrous element of the present invention include dissolution, melting, swelling, shrinking, breaking into pieces, exploding, lengthening, shortening, and combinations thereof. The fibrous elements of the present invention may completely or substantially lose their fibrous element physical structure or they may have their morphology changed or they may retain or substantially retain their fibrous element physical structure as they are exposed to conditions of intended use.

“By weight on a dry fibrous element basis and/or dry soluble fibrous structure basis” means that the weight of the fibrous element and/or soluble fibrous structure measured immediately after the fibrous element and/or soluble fibrous structure has been conditioned in a conditioned room at a temperature of 23° C.±1° C. and a relative humidity of 50%±2% for 2 hours. In one example, “by weight on a dry fibrous element basis and/or dry soluble fibrous structure basis” means that the fibrous element and/or soluble fibrous structure comprises less than 20% and/or less than 15% and/or less than 10% and/or less than 7% and/or less than 5% and/or less than 3% and/or to 0% and/or to greater than 0% based on the weight of the fibrous element and/or soluble fibrous structure of moisture, such as water, for example free water, as measured according to the Water Content Test Method described herein.

“Total level” as used herein, for example with respect to the total level of one or more active agents present in the fibrous element and/or soluble fibrous structure, means the sum of the weights or weight percent of all of the subject materials, for example active agents. In other words, a fibrous element and/or soluble fibrous structure may comprise 25% by weight on a dry fibrous element basis and/or dry soluble fibrous structure basis of an anionic surfactant, 15% by weight on a dry fibrous element basis and/or dry soluble fibrous structure basis of a nonionic surfactant, 10% by weight of a chelant, and 5% of a perfume so that the total level of active agents present in the fibrous element is greater than 50%; namely 55% by weight on a dry fibrous element basis and/or dry soluble fibrous structure basis.

“Detergent product” as used herein means a solid form, for example a rectangular solid, sometimes referred to as a sheet, that comprises one or more active agents, for example a fabric care active agent, a dishwashing active agent, a hard surface active agent, and mixtures thereof. In one example, a detergent product of the present invention comprises one or more surfactants, one or more enzymes, one or more perfumes and/or one or more suds suppressors. In another example, a detergent product of the present invention comprises a builder and/or a chelating agent. In another example, a detergent product of the present invention comprises a bleaching agent.

In one example, the detergent product comprises a web, for example a soluble fibrous structure.

“Web” as used herein means a collection of formed fibrous elements (fibers and/or filaments), such as a fibrous structure, and/or a detergent product formed of fibers and/or filaments, such as continuous filaments, of any nature or origin associated with one another. In one example, the web is a rectangular solid comprising fibers and/or filaments that are formed via a spinning process, not a casting process.

“Particulates” as used herein means granular substances and/or powders. In one example, the filaments and/or fibers can be converted into powders.

“Different from” or “different” as used herein means, with respect to a material, such as a fibrous element as a whole and/or a fibrous element-forming material within a fibrous element and/or an active agent within a fibrous element, that one material, such as a fibrous element and/or a fibrous element-forming material and/or an active agent, is chemically, physically and/or structurally different from another material, such as a fibrous element and/or a fibrous element-forming material and/or an active agent. For example, a fibrous element-forming material in the form of a filament is different from the same fibrous element-forming material in the form of a fiber. Likewise, starch is different from cellulose. However, different molecular weights of the same material, such as different molecular weights of a starch, are not different materials from one another for purposes of the present invention.

“Random mixture of polymers” as used herein means that two or more different fibrous element-forming materials are randomly combined to form a fibrous element. Accordingly, two or more different fibrous element-forming materials that are orderly combined to form a fibrous element, such as a core and sheath bicomponent fibrous element, is not a random mixture of different fibrous element-forming materials for purposes of the present invention.

“Associate,” “Associated,” “Association,” and/or “Associating” as used herein with respect to fibrous elements and/or particle means combining, either in direct contact or in indirect contact, fibrous elements and/or particles such that a fibrous structure is formed. In one example, the associated fibrous elements and/or particles may be bonded together for example by adhesives and/or thermal bonds. In another example, the fibrous elements and/or particles may be associated with one another by being deposited onto the same fibrous structure making belt and/or patterned belt.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Soluble Fibrous Structure

The soluble fibrous structure of the present invention comprises a plurality of fibrous elements, for example a plurality of filaments. In one example, the plurality of fibrous elements is inter-entangled to form a soluble fibrous structure.

In one example of the present invention, the soluble fibrous structure is a water-soluble fibrous structure.

In another example of the present invention, the soluble fibrous structure is an apertured fibrous structure.

Even though the fibrous element and/or soluble fibrous structure of the present invention are in solid form, the fibrous element-forming composition used to make the fibrous elements of the present invention may be in the form of a liquid.

In one example, the soluble fibrous structure comprises a plurality of identical or substantially identical from a compositional perspective of fibrous elements according to the present invention. In another example, the soluble fibrous structure may comprise two or more different fibrous elements according to the present invention. Non-limiting examples of differences in the fibrous elements may be physical differences such as differences in diameter, length, texture, shape, rigidness, elasticity, and the like; chemical differences such as crosslinking level, solubility, melting point, Tg, active agent, fibrous element-forming material, color, level of active agent, basis weight, level of fibrous element-forming material, presence of any coating on fibrous element, biodegradable or not, hydrophobic or not, contact angle, and the like; differences in whether the fibrous element loses its physical structure when the fibrous element is exposed to conditions of intended use; differences in whether the fibrous element's morphology changes when the fibrous element is exposed to conditions of intended use; and differences in rate at which the fibrous element releases one or more of its active agents when the fibrous element is exposed to conditions of intended use. In one example, two or more fibrous elements and/or particles within the soluble fibrous structure may comprise different active agents. This may be the case where the different active agents may be incompatible with one another, for example an anionic surfactant (such as a shampoo active agent) and a cationic surfactant (such as a hair conditioner active agent). In one example, the surfactant is selected from the group consisting of: anionic surfactants, cationic surfactants, nonionic surfactants, zwitterionic surfactants, amphoteric surfactants, and mixtures thereof.

In another example, the soluble fibrous structure may exhibit different regions, such as different regions of basis weight, density, and/or caliper. In yet another example, the soluble fibrous structure may comprise texture on one or more of its surfaces. A surface of the soluble fibrous structure may comprise a pattern, such as a non-random, repeating pattern. The soluble fibrous structure may be embossed with an emboss pattern.

In one example, the water-soluble soluble fibrous structure is a water-soluble fibrous structure comprising a plurality of apertures. The apertures may be arranged in a non-random, repeating pattern.

Apertures within the apertured, water-soluble fibrous structure may be of virtually any shape and size. In one example, the apertures within the apertured, water-soluble fibrous structures are generally round or oblong shaped, in a regular pattern of spaced apart openings. The apertures can each have a diameter of from about 0.1 to about 2 mm and/or from about 0.5 to about 1 mm. The apertures may form an open area within an apertured, water-soluble fibrous structure of from about 0.5% to about 25% and/or from about 1% to about 20% and/or from about 2% to about 10%. It is believed that the benefits of the present invention can be realized with non-repeating and/or non-regular patterns of apertures having various shapes and sizes.

In another example, the fibrous structure may comprise apertures. The apertures may be arranged in a non-random, repeating pattern. Aperturing of fibrous structures, for example water-soluble fibrous structures, can be accomplished by any number of techniques. For example, aperturing can be accomplished by various processes involving bonding and stretching, such as those described in U.S. Pat. Nos. 3,949,127 and 5,873,868. In one embodiment, the apertures may be formed by forming a plurality of spaced, melt stabilized regions, and then ring-rolling the web to stretch the web and form apertures in the melt stabilized regions, as described in U.S. Pat. Nos. 5,628,097 and 5,916,661, both of which are hereby incorporated by reference herein. In another embodiment, apertures can be formed in a multilayer, fibrous structure configuration by the method described in U.S. Pat. Nos. 6,830,800 and 6,863,960 which are hereby incorporated herein by reference. Still another process for aperturing webs is described in U.S. Pat. No. 8,241,543 entitled “Method And Apparatus For Making An Apertured Web”, which is hereby incorporated herein by reference.

In one example, the soluble fibrous structure may comprise discrete regions of fibrous elements that differ from other parts of the soluble fibrous structure.

The soluble fibrous structure of the present invention may be used as is or may be coated with one or more active agents.

In one example, the soluble fibrous structure of the present invention exhibits a thickness of greater than 0.01 mm and/or greater than 0.05 mm and/or greater than 0.1 mm and/or to about 100 mm and/or to about 50 mm and/or to about 20 mm and/or to about 10 mm and/or to about 5 mm and/or to about 2 mm and/or to about 0.5 mm and/or to about 0.3 mm as measured by the Thickness Test Method described herein.

In another example, the soluble fibrous structure of the present invention exhibits a Geometric Mean (GM) Tensile Strength of about 200 g/cm or more, and/or about 500 g/cm or more, and/or about 1000 g/cm or more, and/or about 1500 g/cm or more, and/or about 2000 g/cm or more and/or less than 5000 g/cm and/or less than 4000 g/cm and/or less than 3000 g/cm and/or less than 2500 g/cm as measured according to the Tensile Test Method described herein.

In another example, the soluble fibrous structure of the present invention exhibits a Geometric Mean (GM) Peak Elongation of less than 1000% and/or less than 800% and/or less than 650% and/or less than 550% and/or less than 500% and/or less than 250% and/or less than 100% as measured according to the Tensile Test Method described herein.

In another example, the soluble fibrous structure of the present invention exhibits a Geometric Mean (GM) Tangent Modulus of less than 5000 g/cm and/or less than 3000 g/cm and/or greater than 100 g/cm and/or greater than 500 g/cm and/or greater than 1000 g/cm and/or greater than 1500 g/cm as measured according to the Tensile Test Method described herein.

In another example, the soluble fibrous structure of the present invention exhibits a Geometric Mean (GM) Secant Modulus of less than less than 5000 g/cm and/or less than 3000 g/cm and/or less than 2500 g/cm and/or less than 2000 g/cm and/or less than 1500 g/cm and/or greater than 100 g/cm and/or greater than 300 g/cm and/or greater than 500 g/cm as measured according to the Tensile Test Method described herein.

One or more, and/or a plurality of fibrous elements of the present invention may form a soluble fibrous structure by any suitable process known in the art. The soluble fibrous structure may be used to deliver the active agents from the fibrous elements of the present invention when the soluble fibrous structure is exposed to conditions of intended use of the fibrous elements and/or soluble fibrous structure.

In one example, the soluble fibrous structure comprises a plurality of identical or substantially identical from a compositional perspective fibrous elements according to the present invention. In another example, the soluble fibrous structure may comprise two or more different fibrous elements according to the present invention. Non-limiting examples of differences in the fibrous elements may be physical differences such as differences in diameter, length, texture, shape, rigidness, elasticity, and the like; chemical differences such as crosslinking level, solubility, melting point, Tg, active agent, fibrous element-forming material, color, level of active agent, level of fibrous element-forming material, presence of any coating on fibrous element, biodegradable or not, hydrophobic or not, contact angle, and the like; differences in whether the fibrous element loses its physical structure when the fibrous element is exposed to conditions of intended use; differences in whether the fibrous element's morphology changes when the fibrous element is exposed to conditions of intended use; and differences in rate at which the fibrous element releases one or more of its active agents when the fibrous element is exposed to conditions of intended use. In one example, two or more fibrous elements within the soluble fibrous structure may comprise the same fibrous element-forming material, but have different active agents. This may be the case where the different active agents may be incompatible with one another, for example an anionic surfactant (such as a shampoo active agent) and a cationic surfactant (such as a hair conditioner active agent).

In another example, as shown in FIG. 2, a soluble fibrous structure 14 of the present invention may comprise two or more different layers 16, 18 (in the z-direction of the soluble fibrous structure 14) of fibrous elements 10, for example filaments, of the present invention that form the soluble fibrous structure 14. The fibrous elements 10 in layer 16 may be the same as or different from the fibrous elements 10 of layer 18. Each layer 16, 18 may comprise a plurality of identical or substantially identical or different fibrous elements 10. For example, fibrous elements 10 that may release their active agents at a faster rate than others within the soluble fibrous structure 14 may be positioned to an external surface of the soluble fibrous structure 14.

In another example, the soluble fibrous structure may exhibit different regions, such as different regions of basis weight, density and/or caliper. In yet another example, the soluble fibrous structure may comprise texture on one or more of its surfaces. A surface of the soluble fibrous structure may comprise a pattern, such as a non-random, repeating pattern. The soluble fibrous structure may be embossed with an emboss pattern. In another example, the soluble fibrous structure may comprise apertures. The apertures may be arranged in a non-random, repeating pattern.

In one example, the soluble fibrous structure may comprise discrete regions of fibrous elements that differ from other parts of the soluble fibrous structure. Non-limiting examples of different regions within soluble fibrous structures are described in U.S. Published Patent Application Nos. 2013/017421 and 2013/0167305 incorporated herein by reference.

Non-limiting examples of use of the soluble fibrous structure of the present invention include, but are not limited to a laundry dryer substrate, washing machine substrate, washcloth, hard surface cleaning and/or polishing substrate, floor cleaning and/or polishing substrate, as a component in a battery, baby wipe, adult wipe, feminine hygiene wipe, bath tissue wipe, window cleaning substrate, oil containment and/or scavenging substrate, insect repellant substrate, swimming pool chemical substrate, food, breath freshener, deodorant, waste disposal bag, packaging film and/or wrap, wound dressing, medicine delivery, building insulation, crops and/or plant cover and/or bedding, glue substrate, skin care substrate, hair care substrate, air care substrate, water treatment substrate and/or filter, toilet bowl cleaning substrate, candy substrate, pet food, livestock bedding, teeth whitening substrates, carpet cleaning substrates, and other suitable uses of the active agents of the present invention.

The soluble fibrous structure of the present invention may be used as is or may be coated with one or more active agents.

In another example, the soluble fibrous structure of the present invention may be pressed into a film, for example by applying a compressive force and/or heating the soluble fibrous structure to convert the soluble fibrous structure into a film. The film would comprise the active agents that were present in the fibrous elements of the present invention. The soluble fibrous structure may be completely converted into a film or parts of the soluble fibrous structure may remain in the film after partial conversion of the soluble fibrous structure into the film. The films may be used for any suitable purposes that the active agents may be used for including, but not limited to the uses exemplified for the soluble fibrous structure.

In one example, a soluble fibrous structure of the present invention can exhibit an average disintegration time of about 60 seconds (s) or less, and/or about 30 s or less, and/or about 10 s or less, and/or about 5 s or less, and/or about 2.0 s or less and/or about 1.5 s or less as measured according to the Dissolution Test Method described herein.

In one example, a soluble fibrous structure of the present invention can exhibit an average dissolution time of about 600 seconds (s) or less, and/or about 400 s or less, and/or about 300 s or less, and/or about 200 s or less, and/or about 175 s or less and/or about 100 or less and/or about 50 or less and/or greater than 1 as measured according to the Dissolution Test Method described herein.

In one example, a soluble fibrous structure of the present invention can exhibit an average disintegration time per gsm of sample of about 1.0 second/gsm (s/gsm) or less, and/or about 0.5 s/gsm or less, and/or about 0.2 s/gsm or less, and/or about 0.1 s/gsm or less, and/or about 0.05 s/gsm or less, and/or about 0.03 s/gsm or less as measured according to the Dissolution Test Method described herein.

In one example, a soluble fibrous structure of the present invention having such fibrous elements can exhibit an average dissolution time per gsm of sample of about 10 seconds/gsm (s/gsm) or less, and/or about 5.0 s/gsm or less, and/or about 3.0 s/gsm or less, and/or about 2.0 s/gsm or less, and/or about 1.8 s/gsm or less, and/or about 1.5 s/gsm or less as measured according to the Dissolution Test Method described herein.

In one example, the soluble fibrous structure of the present invention exhibits a thickness of greater than 0.01 mm and/or greater than 0.05 mm and/or greater than 0.1 mm and/or to about 20 mm and/or to about 10 mm and/or to about 5 mm and/or to about 2 mm and/or to about 0.5 mm and/or to about 0.3 mm as measured by the Thickness Test Method described herein.

In certain embodiments, suitable fibrous structures can have a water content (% moisture) from 0% to about 20%; in certain embodiments, fibrous structures can have a water content from about 1% to about 15%; and in certain embodiments, fibrous structures can have a water content from about 5% to about 10% as measured according to the Water Content Test Method described herein.

In one example, the soluble fibrous structure exhibits an Initial Water Propagation Rate of greater than about 5.0×10⁻⁴ m/s and/or greater than about 7.75×10⁻⁴ m/s and/or greater than about 1.0×10⁻³ m/s and/or greater than about 2.0×10⁻³ m/s and/or greater than about 5.0×10⁻³ m/s and/or greater than about 1.0×10⁻² m/s and/or greater than about 2.0×10⁻² m/s and/or greater than about 3.5×10⁻² m/s as measure according to the Initial Water Propagation Rate Test Method described herein.

### Fibrous Elements

The fibrous element, such as a filament and/or fiber, of the present invention comprises one or more fibrous element-forming materials. In addition to the fibrous element-forming materials, the fibrous element may further comprise one or more active agents present within the fibrous element that are releasable from the fibrous element, for example a filament, such as when the fibrous element and/or soluble fibrous structure comprising the fibrous element is exposed to conditions of intended use. In one example, the total level of the one or more fibrous element-forming materials present in the fibrous element is less than 80% by weight on a dry fibrous element basis and/or dry soluble fibrous structure basis and the total level of the one or more active agents present in the fibrous element is greater than 20% by weight on a dry fibrous element basis and/or dry soluble fibrous structure basis.

In one example, the fibrous element of the present invention comprises about 100% and/or greater than 95% and/or greater than 90% and/or greater than 85% and/or greater than 75% and/or greater than 50% by weight on a dry fibrous element basis and/or dry soluble fibrous structure basis of one or more fibrous element-forming materials. For example, the fibrous element-forming material may comprise polyvinyl alcohol, starch, carboxymethylcellulose, and other suitable polymers, especially hydroxyl polymers.

In another example, the fibrous element of the present invention comprises one or more fibrous element-forming materials and one or more active agents wherein the total level of fibrous element-forming materials present in the fibrous element is from about 5% to less than 80% by weight on a dry fibrous element basis and/or dry soluble fibrous structure basis and the total level of active agents present in the fibrous element is greater than 20% to about 95% by weight on a dry fibrous element basis and/or dry soluble fibrous structure basis.

In one example, the fibrous element of the present invention comprises at least 10% and/or at least 15% and/or at least 20% and/or less than less than 80% and/or less than 75% and/or less than 65% and/or less than 60% and/or less than 55% and/or less than 50% and/or less than 45% and/or less than 40% by weight on a dry fibrous element basis and/or dry soluble fibrous structure basis of the fibrous element-forming materials and greater than 20% and/or at least 35% and/or at least 40% and/or at least 45% and/or at least 50% and/or at least 60% and/or less than 95% and/or less than 90% and/or less than 85% and/or less than 80% and/or less than 75% by weight on a dry fibrous element basis and/or dry soluble fibrous structure basis of active agents.

In one example, the fibrous element of the present invention comprises at least 5% and/or at least 10% and/or at least 15% and/or at least 20% and/or less than 50% and/or less than 45% and/or less than 40% and/or less than 35% and/or less than 30% and/or less than 25% by weight on a dry fibrous element basis and/or dry soluble fibrous structure basis of the fibrous element-forming materials and greater than 50% and/or at least 55% and/or at least 60% and/or at least 65% and/or at least 70% and/or less than 95% and/or less than 90% and/or less than 85% and/or less than 80% and/or less than 75% by weight on a dry fibrous element basis and/or dry soluble fibrous structure basis of active agents. In one example, the fibrous element of the present invention comprises greater than 80% by weight on a dry fibrous element basis and/or dry soluble fibrous structure basis of active agents.

In another example, the one or more fibrous element-forming materials and active agents are present in the fibrous element at a weight ratio of total level of fibrous element-forming materials to active agents of 4.0 or less and/or 3.5 or less and/or 3.0 or less and/or 2.5 or less and/or 2.0 or less and/or 1.85 or less and/or less than 1.7 and/or less than 1.6 and/or less than 1.5 and/or less than 1.3 and/or less than 1.2 and/or less than 1 and/or less than 0.7 and/or less than 0.5 and/or less than 0.4 and/or less than 0.3 and/or greater than 0.1 and/or greater than 0.15 and/or greater than 0.2.

In still another example, the fibrous element of the present invention comprises from about 10% and/or from about 15% to less than 80% by weight on a dry fibrous element basis and/or dry soluble fibrous structure basis of a fibrous element-forming material, such as polyvinyl alcohol polymer, starch polymer, and/or carboxymethylcellulose polymer, and greater than 20% to about 90% and/or to about 85% by weight on a dry fibrous element basis and/or dry soluble fibrous structure basis of an active agent. The fibrous element may further comprise a plasticizer, such as glycerin and/or pH adjusting agents, such as citric acid.

In yet another example, the fibrous element of the present invention comprises from about 10% and/or from about 15% to less than 80% by weight on a dry fibrous element basis and/or dry soluble fibrous structure basis of a fibrous element-forming material, such as polyvinyl alcohol polymer, starch polymer, and/or carboxymethylcellulose polymer, and greater than 20% to about 90% and/or to about 85% by weight on a dry fibrous element basis and/or dry soluble fibrous structure basis of an active agent, wherein the weight ratio of fibrous element-forming material to active agent is 4.0 or less. The fibrous element may further comprise a plasticizer, such as glycerin and/or pH adjusting agents, such as citric acid.

In even another example of the present invention, a fibrous element comprises one or more fibrous element-forming materials and one or more active agents selected from the group consisting of: enzymes, bleaching agents, builder, chelants, sensates, dispersants, and mixtures thereof that are releasable and/or released when the fibrous element and/or soluble fibrous structure comprising the fibrous element is exposed to conditions of intended use. In one example, the fibrous element comprises a total level of fibrous element-forming materials of less than 95% and/or less than 90% and/or less than 80% and/or less than 50% and/or less than 35% and/or to about 5% and/or to about 10% and/or to about 20% by weight on a dry fibrous element basis and/or dry soluble fibrous structure basis and a total level of active agents selected from the group consisting of: enzymes, bleaching agents, builder, chelants, perfumes, antimicrobials, antibacterials, antifungals, and mixtures thereof of greater than 5% and/or greater than 10% and/or greater than 20% and/or greater than 35% and/or greater than 50% and/or greater than 65% and/or to about 95% and/or to about 90% and/or to about 80% by weight on a dry fibrous element basis and/or dry soluble fibrous structure basis. In one example, the active agent comprises one or more enzymes. In another example, the active agent comprises one or more bleaching agents. In yet another example, the active agent comprises one or more builders. In still another example, the active agent comprises one or more chelants. In still another example, the active agent comprises one or more perfumes. In even still another example, the active agent comprises one or more antimicrobials, antibacterials, and/or antifungals.

In yet another example of the present invention, the fibrous elements of the present invention may comprise active agents that may create health and/or safety concerns if they become airborne. For example, the fibrous element may be used to inhibit enzymes within the fibrous element from becoming airborne.

In one example, the fibrous elements of the present invention may be meltblown fibrous elements. In another example, the fibrous elements of the present invention may be spunbond fibrous elements. In another example, the fibrous elements may be hollow fibrous elements prior to and/or after release of one or more of its active agents.

The fibrous elements of the present invention may be hydrophilic or hydrophobic. The fibrous elements may be surface treated and/or internally treated to change the inherent hydrophilic or hydrophobic properties of the fibrous element.

In one example, the fibrous element exhibits a diameter of less than 100 μm and/or less than 75 μm and/or less than 50 μm and/or less than 25 μm and/or less than 10 μm and/or less than 5 μm and/or less than 1 μm as measured according to the Diameter Test Method described herein. In another example, the fibrous element of the present invention exhibits a diameter of greater than 1 μm as measured according to the Diameter Test Method described herein. The diameter of a fibrous element of the present invention may be used to control the rate of release of one or more active agents present in the fibrous element and/or the rate of loss and/or altering of the fibrous element's physical structure.

The fibrous element may comprise two or more different active agents. In one example, the fibrous element comprises two or more different active agents, wherein the two or more different active agents are compatible with one another. In another example, the fibrous element comprises two or more different active agents, wherein the two or more different active agents are incompatible with one another.

In one example, the fibrous element may comprise an active agent within the fibrous element and an active agent on an external surface of the fibrous element, such as an active agent coating on the fibrous element, for example a coating composition comprising one or more active agents. The active agent on the external surface of the fibrous element may be the same or different from the active agent present in the fibrous element. If different, the active agents may be compatible or incompatible with one another.

In one example, one or more active agents may be uniformly distributed or substantially uniformly distributed throughout the fibrous element. In another example, one or more active agents may be distributed as discrete regions within the fibrous element. In still another example, at least one active agent is distributed uniformly or substantially uniformly throughout the fibrous element and at least one other active agent is distributed as one or more discrete regions within the fibrous element. In still yet another example, at least one active agent is distributed as one or more discrete regions within the fibrous element and at least one other active agent is distributed as one or more discrete regions different from the first discrete regions within the fibrous element.

In one example, one or more fibrous elements of the soluble fibrous structure of the present invention exhibits a Hydration Value of greater than about 7.75×10⁻⁵ m/s^{1/2} and/or greater than about 9.0×10⁻⁵ m/s^{1/2} and/or greater than about 1.0×10⁻⁴ m/s^{1/2} and/or greater than about 1.25×10⁻⁴ m/s^{1/2} and/or greater than about 1.5×10⁻⁴ m/s^{1/2} and/or less than about 1.0 m/s^{1/2} and/or less than about 1.0×10⁻¹ m/s^{1/2} as measured according to the Hydration Value Test Method described herein.

In another example, one or more fibrous elements of the soluble fibrous structure of the present invention exhibits a Swelling Value of less than about 2.05 and/or less than about 2.0 and/or less than about 1.8 and/or less than about 1.7 and/or less than about 1.5 and/or greater than about 0.5 and/or greater than about 0.75 and/or greater than about 1.0 as measured according to the Swelling Rate Test Method described herein.

In yet another example, one or more fibrous elements of the soluble fibrous structure of the present invention exhibits a Viscosity Value of less than about 100 Pa·s and/or less than about 80 Pa·s and/or less than about 60 Pa·s and/or less than about 40 Pa·s and/or less than about 20 Pa·s and/or less than about 10 Pa·s and/or less than about 5 Pa·s and/or less than about 2 Pa·s and/or less than about 1 Pa·s and/or greater than 0 Pa·s as measured according to the Viscosity Value Test Method described herein.

### Fibrous Element-Forming Material

The fibrous element-forming material is any suitable material, such as a polymer or monomers capable of producing a polymer that exhibits properties suitable for making a fibrous element, such as by a spinning process.

In one example, the fibrous element-forming material may comprise a polar solvent-soluble material, such as an alcohol-soluble material and/or a water-soluble material.

In another example, the fibrous element-forming material may comprise a non-polar solvent-soluble material.

In still another example, the filament forming material may comprise a polar solvent-soluble material and be free (less than 5% and/or less than 3% and/or less than 1% and/or 0% by weight on a dry fibrous element basis and/or dry soluble fibrous structure basis) of non-polar solvent-soluble materials.

In yet another example, the fibrous element-forming material may be a film-forming material. In still yet another example, the fibrous element-forming material may be synthetic or of natural origin and it may be chemically, enzymatically, and/or physically modified.

In even another example of the present invention, the fibrous element-forming material may comprise a polymer selected from the group consisting of: polymers derived from acrylic monomers such as the ethylenically unsaturated carboxylic monomers and ethylenically unsaturated monomers, polyvinyl alcohol, polyacrylates, polymethacrylates, copolymers of acrylic acid and methyl acrylate, polyvinylpyrrolidones, polyalkylene oxides, starch and starch derivatives, pullulan, gelatin, hydroxypropylmethylcelluloses, methycelluloses, and carboxymethycelluloses.

In still another example, the fibrous element-forming material may comprises a polymer selected from the group consisting of: polyvinyl alcohol, polyvinyl alcohol derivatives, starch, starch derivatives, cellulose derivatives, hemicellulose, hemicellulose derivatives, proteins, sodium alginate, hydroxypropyl methylcellulose, chitosan, chitosan derivatives, polyethylene glycol, tetramethylene ether glycol, polyvinyl pyrrolidone, hydroxymethyl cellulose, hydroxyethyl cellulose, and mixtures thereof.

In another example, the fibrous element-forming material comprises a polymer is selected from the group consisting of: pullulan, hydroxypropylmethyl cellulose, hydroxyethyl cellulose, hydroxypropyl cellulose, polyvinyl pyrrolidone, carboxymethyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, and mixtures thereof.

### Polar Solvent-Soluble Materials

Non-limiting examples of polar solvent-soluble materials include polar solvent-soluble polymers. The polar solvent-soluble polymers may be synthetic or natural original and may be chemically and/or physically modified. In one example, the polar solvent-soluble polymers exhibit a weight average molecular weight of at least 10,000 g/mol and/or at least 20,000 g/mol and/or at least 40,000 g/mol and/or at least 80,000 g/mol and/or at least 100,000 g/mol and/or at least 1,000,000 g/mol and/or at least 3,000,000 g/mol and/or at least 10,000,000 g/mol and/or at least 20,000,000 g/mol and/or to about 40,000,000 g/mol and/or to about 30,000,000 g/mol.

In one example, the polar solvent-soluble polymers are selected from the group consisting of: alcohol-soluble polymers, water-soluble polymers and mixtures thereof. Non-limiting examples of water-soluble polymers include water-soluble hydroxyl polymers, water-soluble thermoplastic polymers, water-soluble biodegradable polymers, water-soluble non-biodegradable polymers and mixtures thereof. In one example, the water-soluble polymer comprises polyvinyl alcohol. In another example, the water-soluble polymer comprises starch. In yet another example, the water-soluble polymer comprises polyvinyl alcohol and starch.

a. Water-Soluble Hydroxyl Polymers—

Non-limiting examples of water-soluble hydroxyl polymers in accordance with the present invention include polyols, such as polyvinyl alcohol, polyvinyl alcohol derivatives, polyvinyl alcohol copolymers, starch, starch derivatives, starch copolymers, chitosan, chitosan derivatives, chitosan copolymers, cellulose derivatives such as cellulose ether and ester derivatives, cellulose copolymers, hemicellulose, hemicellulose derivatives, hemicellulose copolymers, gums, arabinans, galactans, proteins and various other polysaccharides and mixtures thereof.

In one example, a water-soluble hydroxyl polymer of the present invention comprises a polysaccharide.

“Polysaccharides” as used herein means natural polysaccharides and polysaccharide derivatives and/or modified polysaccharides. Suitable water-soluble polysaccharides include, but are not limited to, starches, starch derivatives, chitosan, chitosan derivatives, cellulose derivatives, hemicellulose, hemicellulose derivatives, gums, arabinans, galactans and mixtures thereof. The water-soluble polysaccharide may exhibit a weight average molecular weight of from about 10,000 to about 40,000,000 g/mol and/or greater than 100,000 g/mol and/or greater than 1,000,000 g/mol and/or greater than 3,000,000 g/mol and/or greater than 3,000,000 to about 40,000,000 g/mol.

The water-soluble polysaccharides may comprise non-cellulose and/or non-cellulose derivative and/or non-cellulose copolymer water-soluble polysaccharides. Such non-cellulose water-soluble polysaccharides may be selected from the group consisting of: starches, starch derivatives, chitosan, chitosan derivatives, hemicellulose, hemicellulose derivatives, gums, arabinans, galactans and mixtures thereof.

In another example, a water-soluble hydroxyl polymer of the present invention comprises a non-thermoplastic polymer.

The water-soluble hydroxyl polymer may have a weight average molecular weight of from about 10,000 g/mol to about 40,000,000 g/mol and/or greater than 100,000 g/mol and/or greater than 1,000,000 g/mol and/or greater than 3,000,000 g/mol and/or greater than 3,000,000 g/mol to about 40,000,000 g/mol. Higher and lower molecular weight water-soluble hydroxyl polymers may be used in combination with hydroxyl polymers having a certain desired weight average molecular weight.

Well known modifications of water-soluble hydroxyl polymers, such as natural starches, include chemical modifications and/or enzymatic modifications. For example, natural starch can be acid-thinned, hydroxy-ethylated, hydroxy-propylated, and/or oxidized. In addition, the water-soluble hydroxyl polymer may comprise dent corn starch.

Naturally occurring starch is generally a mixture of linear amylose and branched amylopectin polymer of D-glucose units. The amylose is a substantially linear polymer of D-glucose units joined by (1,4)-α-D links. The amylopectin is a highly branched polymer of D-glucose units joined by (1,4)-α-D links and (1,6)-α-D links at the branch points. Naturally occurring starch typically contains relatively high levels of amylopectin, for example, corn starch (64-80% amylopectin), waxy maize (93-100% amylopectin), rice (83-84% amylopectin), potato (about 78% amylopectin), and wheat (73-83% amylopectin). Though all starches are potentially useful herein, the present invention is most commonly practiced with high amylopectin natural starches derived from agricultural sources, which offer the advantages of being abundant in supply, easily replenishable and inexpensive.

As used herein, “starch” includes any naturally occurring unmodified starches, modified starches, synthetic starches and mixtures thereof, as well as mixtures of the amylose or amylopectin fractions; the starch may be modified by physical, chemical, or biological processes, or combinations thereof. The choice of unmodified or modified starch for the present invention may depend on the end product desired. In one embodiment of the present invention, the starch or starch mixture useful in the present invention has an amylopectin content from about 20% to about 100%, more typically from about 40% to about 90%, even more typically from about 60% to about 85% by weight of the starch or mixtures thereof.

Suitable naturally occurring starches can include, but are not limited to, corn starch, potato starch, sweet potato starch, wheat starch, sago palm starch, tapioca starch, rice starch, soybean starch, arrow root starch, amioca starch, bracken starch, lotus starch, waxy maize starch, and high amylose corn starch. Naturally occurring starches particularly, corn starch and wheat starch, are the preferred starch polymers due to their economy and availability.

Polyvinyl alcohols herein can be grafted with other monomers to modify its properties. A wide range of monomers has been successfully grafted to polyvinyl alcohol. Non-limiting examples of such monomers include vinyl acetate, styrene, acrylamide, acrylic acid, 2-hydroxyethyl methacrylate, acrylonitrile, 1,3-butadiene, methyl methacrylate, methacrylic acid, maleic acid, itaconic acid, sodium vinylsulfonate, sodium allylsulfonate, sodium methylallyl sulfonate, sodium phenylallylether sulfonate, sodium phenylmethallylether sulfonate, 2-acrylamido-methyl propane sulfonic acid (AMPs), vinylidene chloride, vinyl chloride, vinyl amine and a variety of acrylate esters.

In one example, the water-soluble hydroxyl polymer is selected from the group consisting of: polyvinyl alcohols, hydroxymethylcelluloses, hydroxyethylcelluloses, hydroxypropylmethylcelluloses and mixtures thereof. A non-limiting example of a suitable polyvinyl alcohol includes those commercially available from Sekisui Specialty Chemicals America, LLC (Dallas, Tex.) under the CELVOL® trade name. A non-limiting example of a suitable hydroxypropylmethylcellulose includes those commercially available from the Dow Chemical Company (Midland, Mich.) under the METHOCEL® trade name including combinations with above mentioned hydroxypropylmethylcelluloses.

b. Water-Soluble Thermoplastic Polymers—

Non-limiting examples of suitable water-soluble thermoplastic polymers include thermoplastic starch and/or starch derivatives, polylactic acid, polyhydroxyalkanoate, polycaprolactone, polyesteramides and certain polyesters, and mixtures thereof.

The water-soluble thermoplastic polymers of the present invention may be hydrophilic or hydrophobic. The water-soluble thermoplastic polymers may be surface treated and/or internally treated to change the inherent hydrophilic or hydrophobic properties of the thermoplastic polymer.

The water-soluble thermoplastic polymers may comprise biodegradable polymers.

Any suitable weight average molecular weight for the thermoplastic polymers may be used. For example, the weight average molecular weight for a thermoplastic polymer in accordance with the present invention is greater than about 10,000 g/mol and/or greater than about 40,000 g/mol and/or greater than about 50,000 g/mol and/or less than about 500,000 g/mol and/or less than about 400,000 g/mol and/or less than about 200,000 g/mol.

### Non-Polar Solvent-Soluble Materials

Non-limiting examples of non-polar solvent-soluble materials include non-polar solvent-soluble polymers. Non-limiting examples of suitable non-polar solvent-soluble materials include cellulose, chitin, chitin derivatives, polyolefins, polyesters, copolymers thereof, and mixtures thereof. Non-limiting examples of polyolefins include polypropylene, polyethylene and mixtures thereof. A non-limiting example of a polyester includes polyethylene terephthalate.

The non-polar solvent-soluble materials may comprise a non-biodegradable polymer such as polypropylene, polyethylene and certain polyesters.

Any suitable weight average molecular weight for the thermoplastic polymers may be used. For example, the weight average molecular weight for a thermoplastic polymer in accordance with the present invention is greater than about 10,000 g/mol and/or greater than about 40,000 g/mol and/or greater than about 50,000 g/mol and/or less than about 500,000 g/mol and/or less than about 400,000 g/mol and/or less than about 200,000 g/mol.

### Active Agents

Active agents are a class of additives that are designed and intended to provide a benefit to something other than the fibrous element and/or particle and/or soluble fibrous structure itself, such as providing a benefit to an environment external to the fibrous element and/or particle and/or soluble fibrous structure. Active agents may be any suitable additive that produces an intended effect under intended use conditions of the fibrous element. For example, the active agent may be selected from the group consisting of: personal cleansing and/or conditioning agents such as hair care agents such as shampoo agents and/or hair colorant agents, hair conditioning agents, skin care agents, sunscreen agents, and skin conditioning agents; laundry care and/or conditioning agents such as fabric care agents, fabric conditioning agents, fabric softening agents, fabric anti-wrinkling agents, fabric care anti-static agents, fabric care stain removal agents, soil release agents, dispersing agents, suds suppressing agents, suds boosting agents, anti-foam agents, and fabric refreshing agents; liquid and/or powder dishwashing agents (for hand dishwashing and/or automatic dishwashing machine applications), hard surface care agents, and/or conditioning agents and/or polishing agents; other cleaning and/or conditioning agents such as antimicrobial agents, antibacterial agents, antifungal agents, fabric hueing agents, perfume, bleaching agents (such as oxygen bleaching agents, hydrogen peroxide, percarbonate bleaching agents, perborate bleaching agents, chlorine bleaching agents), bleach activating agents, chelating agents, builders, lotions, brightening agents, air care agents, carpet care agents, dye transfer-inhibiting agents, clay soil removing agents, anti-redeposition agents, polymeric soil release agents, polymeric dispersing agents, alkoxylated polyamine polymers, alkoxylated polycarboxylate polymers, amphilic graft copolymers, dissolution aids, buffering systems, water-softening agents, water-hardening agents, pH adjusting agents, enzymes, flocculating agents, effervescent agents, preservatives, cosmetic agents, make-up removal agents, lathering agents, deposition aid agents, coacervate-forming agents, clays, thickening agents, latexes, silicas, drying agents, odor control agents, antiperspirant agents, cooling agents, warming agents, absorbent gel agents, anti-inflammatory agents, dyes, pigments, acids, and bases; liquid treatment active agents; agricultural active agents; industrial active agents; ingestible active agents such as medicinal agents, teeth whitening agents, tooth care agents, mouthwash agents, periodontal gum care agents, edible agents, dietary agents, vitamins, minerals; water-treatment agents such as water clarifying and/or water disinfecting agents, and mixtures thereof.

Non-limiting examples of suitable cosmetic agents, skin care agents, skin conditioning agents, hair care agents, and hair conditioning agents are described in CTFA Cosmetic Ingredient Handbook, Second Edition, The Cosmetic, Toiletries, and Fragrance Association, Inc. 1988, 1992.

One or more classes of chemicals may be useful for one or more of the active agents listed above. For example, surfactants may be used for any number of the active agents described above. Likewise, bleaching agents may be used for fabric care, hard surface cleaning, dishwashing and even teeth whitening. Therefore, one of ordinary skill in the art will appreciate that the active agents will be selected based upon the desired intended use of the fibrous element and/or particle and/or soluble fibrous structure made therefrom.

For example, if the fibrous element and/or particle and/or soluble fibrous structure made therefrom is to be used for hair care and/or conditioning then one or more suitable surfactants, such as a lathering surfactant could be selected to provide the desired benefit to a consumer when exposed to conditions of intended use of the fibrous element and/or particle and/or soluble fibrous structure incorporating the fibrous element and/or particle.

In one example, if the fibrous element and/or particle and/or soluble fibrous structure made therefrom is designed or intended to be used for laundering clothes in a laundry operation, then one or more suitable surfactants and/or enzymes and/or builders and/or perfumes and/or suds suppressors and/or bleaching agents could be selected to provide the desired benefit to a consumer when exposed to conditions of intended use of the fibrous element and/or particle and/or soluble fibrous structure incorporating the fibrous element and/or particle. In another example, if the fibrous element and/or particle and/or soluble fibrous structure made therefrom is designed to be used for laundering clothes in a laundry operation and/or cleaning dishes in a dishwashing operation, then the fibrous element and/or particle and/or soluble fibrous structure may comprise a laundry detergent composition or dishwashing detergent composition or active agents used in such compositions. In still another example, if the fibrous element and/or particle and/or soluble fibrous structure made therefrom is designed to be used for cleaning and/or sanitizing a toilet bowl, then the fibrous element and/or particle and/or soluble fibrous structure made therefrom may comprise a toilet bowl cleaning composition and/or effervescent composition and/or active agents used in such compositions.

In one example, the active agent is selected from the group consisting of: surfactants, bleaching agents, enzymes, suds suppressors, suds boosting agents, fabric softening agents, denture cleaning agents, hair cleaning agents, hair care agents, personal health care agents, hueing agents, and mixtures thereof.

### Release of Active Agent

One or more active agents may be released from the fibrous element and/or particle and/or soluble fibrous structure when the fibrous element and/or particle and/or soluble fibrous structure are exposed to a triggering condition. In one example, one or more active agents may be released from the fibrous element and/or particle and/or soluble fibrous structure or a part thereof when the fibrous element and/or particle and/or soluble fibrous structure or the part thereof loses its identity, in other words, loses its physical structure. For example, a fibrous element and/or particle and/or soluble fibrous structure loses its physical structure when the fibrous element-forming material dissolves, melts or undergoes some other transformative step such that its structure is lost. In one example, the one or more active agents are released from the fibrous element and/or particle and/or soluble fibrous structure when the fibrous element's and/or particle's and/or soluble fibrous structure's morphology changes.

In another example, one or more active agents may be released from the fibrous element and/or particle and/or soluble fibrous structure or a part thereof when the fibrous element and/or particle and/or soluble fibrous structure or the part thereof alters its identity, in other words, alters its physical structure rather than loses its physical structure. For example, a fibrous element and/or particle and/or soluble fibrous structure alters its physical structure when the fibrous element-forming material swells, shrinks, lengthens, and/or shortens, but retains its fibrous element-forming properties.

In another example, one or more active agents may be released from the fibrous element and/or particle and/or soluble fibrous structure with its morphology not changing (not losing or altering its physical structure).

In one example, the fibrous element and/or particle and/or soluble fibrous structure may release an active agent upon the fibrous element and/or particle and/or soluble fibrous structure being exposed to a triggering condition that results in the release of the active agent, such as by causing the fibrous element and/or particle and/or soluble fibrous structure to lose or alter its identity as discussed above. Non-limiting examples of triggering conditions include exposing the fibrous element and/or particle and/or soluble fibrous structure to solvent, a polar solvent, such as alcohol and/or water, and/or a non-polar solvent, which may be sequential, depending upon whether the fibrous element-forming material comprises a polar solvent-soluble material and/or a non-polar solvent-soluble material; exposing the fibrous element and/or particle and/or soluble fibrous structure to heat, such as to a temperature of greater than 75° F. and/or greater than 100° F. and/or greater than 150° F. and/or greater than 200° F. and/or greater than 212° F.; exposing the fibrous element and/or particle and/or soluble fibrous structure to cold, such as to a temperature of less than 40° F. and/or less than 32° F. and/or less than 0° F.; exposing the fibrous element and/or particle and/or soluble fibrous structure to a force, such as a stretching force applied by a consumer using the fibrous element and/or particle and/or soluble fibrous structure; and/or exposing the fibrous element and/or particle and/or soluble fibrous structure to a chemical reaction; exposing the fibrous element and/or particle and/or soluble fibrous structure to a condition that results in a phase change; exposing the fibrous element and/or particle and/or soluble fibrous structure to a pH change and/or a pressure change and/or temperature change; exposing the fibrous element and/or particle and/or soluble fibrous structure to one or more chemicals that result in the fibrous element and/or particle and/or soluble fibrous structure releasing one or more of its active agents; exposing the fibrous element and/or particle and/or soluble fibrous structure to ultrasonics; exposing the fibrous element and/or particle and/or soluble fibrous structure to light and/or certain wavelengths; exposing the fibrous element and/or particle and/or soluble fibrous structure to a different ionic strength; and/or exposing the fibrous element and/or particle and/or soluble fibrous structure to an active agent released from another fibrous element and/or particle and/or soluble fibrous structure.

In one example, one or more active agents may be released from the fibrous elements and/or particles of the present invention when a soluble fibrous structure comprising the fibrous elements and/or particles is subjected to a triggering step selected from the group consisting of: pre-treating stains on a fabric article with the soluble fibrous structure; forming a wash liquor by contacting the soluble fibrous structure with water; tumbling the soluble fibrous structure in a dryer; heating the soluble fibrous structure in a dryer; and combinations thereof.

### Fibrous Element-Forming Composition

The fibrous elements of the present invention are made from a fibrous element-forming composition. The fibrous element-forming composition is a polar-solvent-based composition. In one example, the fibrous element-forming composition is an aqueous composition comprising one or more fibrous element-forming materials and one or more active agents.

The fibrous element-forming composition may be processed at a temperature of from about 20° C. to about 100° C. and/or from about 30° C. to about 90° C. and/or from about 35° C. to about 70° C. and/or from about 40° C. to about 60° C. when making fibrous elements from the fibrous element-forming composition.

In one example, the fibrous element-forming composition may comprise at least 20% and/or at least 30% and/or at least 40% and/or at least 45% and/or at least 50% to about 90% and/or to about 85% and/or to about 80% and/or to about 75% by weight of one or more fibrous element-forming materials, one or more active agents, and mixtures thereof. The fibrous element-forming composition may comprise from about 10% to about 80% by weight of a polar solvent, such as water.

In one example, non-volatile components of the fibrous element-forming composition may comprise from about 20% and/or 30% and/or 40% and/or 45% and/or 50% to about 75% and/or 80% and/or 85% and/or 90% by weight based on the total weight of the fibrous element-forming composition. The non-volatile components may be composed of fibrous element-forming materials, such as backbone polymers, active agents and combinations thereof. Volatile components of the fibrous element-forming composition will comprise the remaining percentage and range from 10% to 80% by weight based on the total weight of the fibrous element-forming composition.

In a fibrous element spinning process, the fibrous elements need to have initial stability as they leave the spinning die. Capillary Number is used to characterize this initial stability criterion. At the conditions of the die, the Capillary Number may be at least 1 and/or at least 3 and/or at least 4 and/or at least 5.

In one example, the fibrous element-forming composition exhibits a Capillary Number of from at least about 1 to about 50 and/or at least about 3 to about 50 and/or at least about 5 to about 30 such that the fibrous element-forming composition can be effectively polymer processed into a fibrous element.

“Polymer processing” as used herein means any spinning operation and/or spinning process by which a fibrous element comprising a processed fibrous element-forming material is formed from a fibrous element-forming composition. The spinning operation and/or process may include spun bonding, melt blowing, electro-spinning, rotary spinning, continuous filament producing and/or tow fiber producing operations/processes. A “processed fibrous element-forming material” as used herein means any fibrous element-forming material that has undergone a melt processing operation and a subsequent polymer processing operation resulting in a fibrous element.

The Capillary Number is a dimensionless number used to characterize the likelihood of this droplet breakup. A larger Capillary Number indicates greater fluid stability upon exiting the die. The Capillary Number is defined as follows:

$$Ca = \frac{V * η}{σ}$$

V is the fluid velocity at the die exit (units of Length per Time),
η is the fluid viscosity at the conditions of the die (units of Mass per Length*Time),
σ is the surface tension of the fluid (units of mass per Time²). When velocity, viscosity, and surface tension are expressed in a set of consistent units, the resulting Capillary Number will have no units of its own; the individual units will cancel out.

The Capillary Number is defined for the conditions at the exit of the die. The fluid velocity is the average velocity of the fluid passing through the die opening. The average velocity is defined as follows:

$$V = \frac{{Vol}^{′}}{Area}$$

Vol′=volumetric flowrate (units of Length³ per Time),
Area=cross-sectional area of the die exit (units of Length).

When the die opening is a circular hole, then the fluid velocity can be defined as

$$V = \frac{{Vol}^{′}}{π * {R}^{2}}$$

R is the radius of the circular hole (units of length).

The fluid viscosity will depend on the temperature and may depend of the shear rate. The definition of a shear thinning fluid includes a dependence on the shear rate. The surface tension will depend on the makeup of the fluid and the temperature of the fluid.

In one example, the fibrous element-forming composition may comprise one or more release agents and/or lubricants. Non-limiting examples of suitable release agents and/or lubricants include fatty acids, fatty acid salts, fatty alcohols, fatty esters, sulfonated fatty acid esters, fatty amine acetates and fatty amides, silicones, aminosilicones, fluoropolymers and mixtures thereof.

In one example, the fibrous element-forming composition may comprise one or more antiblocking and/or detackifying agents. Non-limiting examples of suitable antiblocking and/or detackifying agents include starches, modified starches, crosslinked polyvinylpyrrolidone, crosslinked cellulose, microcrystalline cellulose, silica, metallic oxides, calcium carbonate, talc and mica.

Active agents of the present invention may be added to the fibrous element-forming composition prior to and/or during fibrous element formation and/or may be added to the fibrous element after fibrous element formation. For example, a perfume active agent may be applied to the fibrous element and/or soluble fibrous structure comprising the fibrous element after the fibrous element and/or soluble fibrous structure according to the present invention are formed. In another example, an enzyme active agent may be applied to the fibrous element and/or soluble fibrous structure comprising the fibrous element after the fibrous element and/or soluble fibrous structure according to the present invention are formed. In still another example, one or more particles, which may not be suitable for passing through the spinning process for making the fibrous element, may be applied to the fibrous element and/or soluble fibrous structure comprising the fibrous element after the fibrous element and/or soluble fibrous structure according to the present invention are formed.

In one example, the fibrous element-forming composition of the present invention exhibits a Viscosity Value of less than about 100 Pa·s and/or less than about 80 Pa·s and/or less than about 60 Pa·s and/or less than about 40 Pa·s and/or less than about 20 Pa·s and/or less than about 10 Pa·s and/or less than about 5 Pa·s and/or less than about 2 Pa·s and/or less than about 1 Pa·s and/or greater than 0 Pa·s as measured according to the Viscosity Value Test Method described herein.

### Extensional Aids

In one example, the fibrous element comprises an extensional aid. Non-limiting examples of extensional aids can include polymers, other extensional aids, and combinations thereof.

In one example, the extensional aids have a weight-average molecular weight of at least about 500,000 Da. In another example, the weight average molecular weight of the extensional aid is from about 500,000 to about 25,000,000, in another example from about 800,000 to about 22,000,000, in yet another example from about 1,000,000 to about 20,000,000, and in another example from about 2,000,000 to about 15,000,000. The high molecular weight extensional aids are especially suitable in some examples of the invention due to the ability to increase extensional melt viscosity and reducing melt fracture.

The extensional aid, when used in a meltblowing process, is added to the composition of the present invention in an amount effective to visibly reduce the melt fracture and capillary breakage of fibers during the spinning process such that substantially continuous fibers having relatively consistent diameter can be melt spun. Regardless of the process employed to produce fibrous elements and/or particles, the extensional aids, when used, can be present from about 0.001% to about 10%, by weight on a dry fibrous element basis and/or dry particle basis and/or dry soluble fibrous structure basis, in one example, and in another example from about 0.005 to about 5%, by weight on a dry fibrous element basis and/or dry particle basis and/or dry soluble fibrous structure basis, in yet another example from about 0.01 to about 1%, by weight on a dry fibrous element basis and/or dry particle basis and/or dry soluble fibrous structure basis, and in another example from about 0.05% to about 0.5%, by weight on a dry fibrous element basis and/or dry particle basis and/or dry soluble fibrous structure basis.

Non-limiting examples of polymers that can be used as extensional aids can include alginates, carrageenans, pectin, chitin, guar gum, xanthum gum, agar, gum arabic, karaya gum, tragacanth gum, locust bean gum, alkylcellulose, hydroxyalkylcellulose, carboxyalkylcellulose, and mixtures thereof.

Non-limiting examples of other extensional aids can include modified and unmodified polyacrylamide, polyacrylic acid, polymethacrylic acid, polyvinyl alcohol, polyvinylacetate, polyvinylpyrrolidone, polyethylene vinyl acetate, polyethyleneimine, polyamides, polyalkylene oxides including polyethylene oxide, polypropylene oxide, polyethylenepropylene oxide, and mixtures thereof.

### Dissolution Aids

The fibrous elements of the present invention may incorporate dissolution aids to accelerate dissolution when the fibrous element contains more than 40% surfactant to mitigate formation of insoluble or poorly soluble surfactant aggregates that can sometimes form or when the surfactant compositions are used in cold water. Non-limiting examples of dissolution aids include sodium chloride, sodium sulfate, potassium chloride, potassium sulfate, magnesium chloride, and magnesium sulfate.

### Buffer System

The fibrous elements of the present invention may be formulated such that, during use in an aqueous cleaning operation, for example washing clothes or dishes and/or washing hair, the wash water will have a pH of between about 5.0 and about 12 and/or between about 7.0 and 10.5. In the case of a dishwashing operation, the pH of the wash water typically is between about 6.8 and about 9.0. In the case of washing clothes, the pH of the was water typically is between 7 and 11. Techniques for controlling pH at recommended usage levels include the use of buffers, alkalis, acids, etc., and are well known to those skilled in the art. These include the use of sodium carbonate, citric acid or sodium citrate, monoethanol amine or other amines, boric acid or borates, and other pH-adjusting compounds well known in the art.

Fibrous elements and/or soluble fibrous structures useful as “low pH” detergent compositions are included in the present invention and are especially suitable for the surfactant systems of the present invention and may provide in-use pH values of less than 8.5 and/or less than 8.0 and/or less than 7.0 and/or less than 7.0 and/or less than 5.5 and/or to about 5.0.

Dynamic in-wash pH profile fibrous elements are included in the present invention. Such fibrous elements may use wax-covered citric acid particles in conjunction with other pH control agents such that (i) 3 minutes after contact with water, the pH of the wash liquor is greater than 10; (ii) 10 mins after contact with water, the pH of the wash liquor is less than 9.5; (iii) 20 mins after contact with water, the pH of the wash liquor is less than 9.0; and (iv) optionally, wherein, the equilibrium pH of the wash liquor is in the range of from above 7.0 to 8.5.

### Non-Limiting Example of Method for Making Fibrous Elements

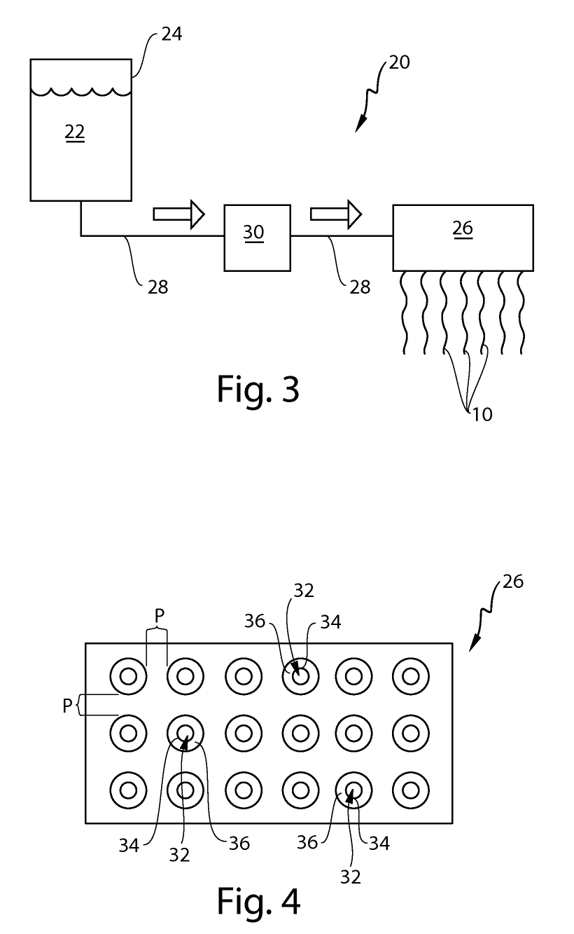

The fibrous elements, for example filaments, of the present invention may be made as shown in FIGS. 3 and 4. As shown in FIGS. 3 and 4, a method 20 for making a fibrous element 10, for example filament, according to the present invention comprises the steps of:

a. providing a fibrous element-forming composition 22, such as from a tank 24, comprising one or more fibrous element-forming materials and one or more active agents; and

b. spinning the fibrous element-forming composition 22, such as via a spinning die 26, into one or more fibrous elements 10, such as filaments, comprising the one or more fibrous element-forming materials and the one or more active agents.

The fibrous element-forming composition may be transported via suitable piping 28, with or without a pump 30, between the tank 24 and the spinning die 26. In one example, a pressurized tank 24, suitable for batch operation is filled with a suitable fibrous element-forming composition 22 for spinning A pump 30, such as a Zenith®, type PEP II, having a capacity of 5.0 cubic centimeters per revolution (cc/rev), manufactured by Colfax Corporation, Zenith Pumps Division, of Monroe, N.C., USA may be used to facilitate transport of the fibrous element-forming composition 22 to a spinning die 26. The flow of the fibrous element-forming composition 22 from the pressurized tank 24 to the spinning die 26 may be controlled by adjusting the number of revolutions per minute (rpm) of the pump 30. Pipes 28 are used to connect the pressurized tank 24, the pump 30, and the spinning die 26 in order to transport (as represented by the arrows) the fibrous element-forming composition 22 from the tank 24 to the pump 30 and into the die 26.

The total level of the one or more fibrous element-forming materials present in the fibrous element 10, when active agents are present therein, may be less than 80% and/or less than 70% and/or less than 65% and/or 50% or less by weight on a dry fibrous element basis and/or dry soluble fibrous structure basis and the total level of the one or more active agents, when present in the fibrous element may be greater than 20% and/or greater than 35% and/or 50% or greater 65% or greater and/or 80% or greater by weight on a dry fibrous element basis and/or dry soluble fibrous structure basis.

As shown in FIGS. 3 and 4, the spinning die 26 may comprise a plurality of fibrous element-forming holes 32 that include a melt capillary 34 encircled by a concentric attenuation fluid hole 36 through which a fluid, such as air, passes to facilitate attenuation of the fibrous element-forming composition 22 into a fibrous element 10 as it exits the fibrous element-forming hole 32.

In one example, the spinning die 26 shown in FIG. 4 has two or more rows of circular extrusion nozzles (fibrous element-forming holes 32) spaced from one another at a pitch P of about 1.524 millimeters (about 0.060 inches). The nozzles have individual inner diameters of about 0.305 millimeters (about 0.012 inches) and individual outside diameters of about 0.813 millimeters (about 0.032 inches). Each individual nozzle comprises a melt capillary 34 encircled by an annular and divergently flared orifice (concentric attenuation fluid hole 36) to supply attenuation air to each individual melt capillary 34. The fibrous element-forming composition 22 extruded through the nozzles is surrounded and attenuated by generally cylindrical, humidified air streams supplied through the orifices to produce fibrous elements 10.

Attenuation air can be provided by heating compressed air from a source by an electrical-resistance heater, for example, a heater manufactured by Chromalox, Division of Emerson Electric, of Pittsburgh, Pa., USA. An appropriate quantity of steam was added to saturate or nearly saturate the heated air at the conditions in the electrically heated, thermostatically controlled delivery pipe. Condensate was removed in an electrically heated, thermostatically controlled, separator.

The embryonic fibrous elements are dried by a drying air stream having a temperature from about 149° C. (about 300° F.) to about 315° C. (about 600° F.) by an electrical resistance heater (not shown) supplied through drying nozzles and discharged at an angle of about 90° relative to the general orientation of the embryonic fibrous elements being spun. The dried fibrous elements may be collected on a collection device, such as a belt or fabric, in one example a belt or fabric capable of imparting a pattern, for example a non-random repeating pattern to a soluble fibrous structure formed as a result of collecting the fibrous elements on the belt or fabric. The addition of a vacuum source directly under the formation zone may be used to aid collection of the fibrous elements on the collection device. The spinning and collection of the fibrous elements produce a soluble fibrous structure comprising inter-entangled fibrous elements, for example filaments.

In one example, during the spinning step, any volatile solvent, such as water, present in the fibrous element-forming composition 22 is removed, such as by drying, as the fibrous element 10 is formed. In one example, greater than 30% and/or greater than 40% and/or greater than 50% of the weight of the fibrous element-forming composition's volatile solvent, such as water, is removed during the spinning step, such as by drying the fibrous element 10 being produced.

The fibrous element-forming composition may comprise any suitable total level of fibrous element-forming materials and any suitable level of active agents so long as the fibrous element produced from the fibrous element-forming composition comprises a total level of fibrous element-forming materials in the fibrous element of from about 5% to 50% or less by weight on a dry fibrous element basis and/or dry particle basis and/or dry soluble fibrous structure basis and a total level of active agents in the fibrous element of from 50% to about 95% by weight on a dry fibrous element basis and/or dry particle basis and/or dry soluble fibrous structure basis.

In one example, the fibrous element-forming composition may comprise any suitable total level of fibrous element-forming materials and any suitable level of active agents so long as the fibrous element produced from the fibrous element-forming composition comprises a total level of fibrous element-forming materials in the fibrous element and/or particle of from about 5% to 50% or less by weight on a dry fibrous element basis and/or dry particle basis and/or dry soluble fibrous structure basis and a total level of active agents in the fibrous element and/or particle of from 50% to about 95% by weight on a dry fibrous element basis and/or dry particle basis and/or dry soluble fibrous structure basis, wherein the weight ratio of fibrous element-forming material to total level of active agents is 1 or less.

In one example, the fibrous element-forming composition comprises from about 1% and/or from about 5% and/or from about 10% to about 50% and/or to about 40% and/or to about 30% and/or to about 20% by weight of the fibrous element-forming composition of fibrous element-forming materials; from about 1% and/or from about 5% and/or from about 10% to about 50% and/or to about 40% and/or to about 30% and/or to about 20% by weight of the fibrous element-forming composition of active agents; and from about 20% and/or from about 25% and/or from about 30% and/or from about 40% and/or to about 80% and/or to about 70% and/or to about 60% and/or to about 50% by weight of the fibrous element-forming composition of a volatile solvent, such as water. The fibrous element-forming composition may comprise minor amounts of other active agents, such as less than 10% and/or less than 5% and/or less than 3% and/or less than 1% by weight of the fibrous element-forming composition of plasticizers, pH adjusting agents, and other active agents.

The fibrous element-forming composition is spun into one or more fibrous elements and/or particles by any suitable spinning process, such as meltblowing, spunbonding, electro-spinning, and/or rotary spinning. In one example, the fibrous element-forming composition is spun into a plurality of fibrous elements and/or particles by meltblowing. For example, the fibrous element-forming composition may be pumped from a tank to a meltblown spinnerette. Upon exiting one or more of the fibrous element-forming holes in the spinnerette, the fibrous element-forming composition is attenuated with air to create one or more fibrous elements and/or particles. The fibrous elements and/or particles may then be dried to remove any remaining solvent used for spinning, such as the water.

The fibrous elements and/or particles of the present invention may be collected on a belt (not shown), such as a patterned belt, for example in an inter-entangled manner such that a soluble fibrous structure comprising the fibrous elements and/or particles is formed.

### Process for Making a Film

The soluble fibrous structure of the present invention may be converted into a film. An example of a process for making a film from a soluble fibrous structure according to the present invention comprises the steps of:

a. providing a soluble fibrous structure comprising a plurality of fibrous elements comprising a fibrous element-forming material, for example a polar solvent-soluble fibrous element-forming material; and

b. converting the soluble fibrous structure into a film.

In one example of the present invention, a process for making a film from a soluble fibrous structure comprises the steps of providing a soluble fibrous structure and converting the soluble fibrous structure into a film.

The step of converting the soluble fibrous structure into a film may comprise the step of subjecting the soluble fibrous structure to a force. The force may comprise a compressive force. The compressive force may apply from about 0.2 MPa and/or from about 0.4 MPa and/or from about 1 MPa and/or to about 10 MPa and/or to about 8 MPa and/or to about 6 MPa of pressure to the soluble fibrous structure.

The soluble fibrous structure may be subjected to the force for at least 20 milliseconds and/or at least 50 milliseconds and/or at least 100 milliseconds and/or to about 800 milliseconds and/or to about 600 milliseconds and/or to about 400 milliseconds and/or to about 200 milliseconds. In one example, the soluble fibrous structure is subjected to the force for a time period of from about 400 milliseconds to about 800 milliseconds.

The soluble fibrous structure may be subjected to the force at a temperature of at least 50° C. and/or at least 100° C. and/or at least 140° C. and/or at least 150° C. and/or at least 180° C. and/or to about 200° C. In one example, the soluble fibrous structure is subjected to the force at a temperature of from about 140° C. to about 200° C.

The soluble fibrous structure may be supplied from a roll of soluble fibrous structure. The resulting film may be wound into a roll of film.

### Methods of Use

In one example, the soluble fibrous structures or films comprising one or more fabric care active agents according the present invention may be utilized in a method for treating a fabric article. The method of treating a fabric article may comprise one or more steps selected from the group consisting of: (a) pre-treating the fabric article before washing the fabric article; (b) contacting the fabric article with a wash liquor formed by contacting the soluble fibrous structure or film with water; (c) contacting the fabric article with the soluble fibrous structure or film in a dryer; (d) drying the fabric article in the presence of the soluble fibrous structure or film in a dryer; and (e) combinations thereof.

In some embodiments, the method may further comprise the step of pre-moistening the soluble fibrous structure or film prior to contacting it to the fabric article to be pre-treated. For example, the soluble fibrous structure or film can be pre-moistened with water and then adhered to a portion of the fabric comprising a stain that is to be pre-treated. Alternatively, the fabric may be moistened and the web or film placed on or adhered thereto. In some embodiments, the method may further comprise the step of selecting of only a portion of the soluble fibrous structure or film for use in treating a fabric article. For example, if only one fabric care article is to be treated, a portion of the soluble fibrous structure or film may be cut and/or torn away and either placed on or adhered to the fabric or placed into water to form a relatively small amount of wash liquor which is then used to pre-treat the fabric. In this way, the user may customize the fabric treatment method according to the task at hand. In some embodiments, at least a portion of a soluble fibrous structure or film may be applied to the fabric to be treated using a device. Exemplary devices include, but are not limited to, brushes and sponges. Any one or more of the aforementioned steps may be repeated to achieve the desired fabric treatment benefit.

In another example, the soluble fibrous structures or films comprising one or more hair care active agents according the present invention may be utilized in a method for treating hair. The method of treating hair may comprise one or more steps selected from the group consisting of: (a) pre-treating the hair before washing the hair; (b) contacting the hair with a wash liquor formed by contacting the soluble fibrous structure or film with water; (c) post-treating the hair after washing the hair; (d) contacting the hair with a conditioning fluid formed by contacting the soluble fibrous structure or film with water; and (e) combinations thereof.

### Methods for Making a Pouch

A pouch comprising a soluble fibrous structure of the present invention may be made by any suitable process known in the art so long as a soluble fibrous structure, for example a water-soluble fibrous structure, of the present invention is used to form at least a portion of the pouch.

In one example, a pouch of the present invention may be made using any suitable equipment and method known in the art. For example, single compartment pouches may be made by vertical and/or horizontal form filling techniques commonly known in the art. Non-limiting examples of suitable processes for making water-soluble pouches, albeit with film wall materials, are described in EP 1504994, EP 2258820, and WO02/40351 (all assigned to The Procter & Gamble Company), which are incorporated herein by reference.

In another example, the process for preparing the pouches of the present invention may comprise the step of shaping pouches from a fibrous structure in a series of molds, wherein the molds are positioned in an interlocking manner. By shaping, it is typically meant that the fibrous structure is placed onto and into the molds, for example, the fibrous structure may be vacuum pulled into the molds, so that the fibrous structure is flush with the inner walls of the molds. This is commonly known as vacuum forming. Another method is thermo-forming to get the fibrous structure to adopt the shape of the mold.

Thermo-forming typically involves the step of formation of an open pouch in a mold under application of heat, which allows the fibrous structure used to make the pouches to take on the shape of the molds.

Vacuum-forming typically involves the step of applying a (partial) vacuum (reduced pressure) on a mold which pulls the fibrous structure into the mold and ensures the fibrous structure adopts the shape of the mold. The pouch forming process may also be done by first heating the fibrous structure and then applying reduced pressure, e.g. (partial) vacuum.

The fibrous structure is typically sealed by any sealing means. For example, by heat sealing, wet sealing or by pressure sealing. In one example, a sealing source is contacted to the fibrous structure and heat or pressure is applied to the fibrous structure, and the fibrous structure is sealed. The sealing source may be a solid object, for example a metal, plastic or wood object. If heat is applied to the fibrous structure during the sealing process, then said sealing source is typically heated to a temperature of from about 40° C. to about 200° C. If pressure is applied to the fibrous structure during the sealing process, then the sealing source typically applies a pressure of from about 1×10⁴ Nm⁻² to about 1×10⁶ Nm⁻², to the fibrous structure.

In another example, the same piece of fibrous structure may be folded, and sealed to form the pouches. Typically more than one piece of fibrous structure is used in the process. For example, a first piece of the fibrous structure may be vacuum pulled into the molds so that the fibrous structure is flush with the inner walls of the molds. A second piece of fibrous structure may be positioned such that it at least partially overlaps and/or completely overlaps, with the first piece of fibrous structure. The first piece of fibrous structure and second piece of fibrous structure are sealed together. The first piece of fibrous structure and second piece of fibrous structure can be the same or different.

In another example of making pouches of the present invention, a first piece of fibrous structure may be vacuum pulled into the molds so that the fibrous structure is flush with the inner walls of the molds. A composition, such as one or more active agents and/or a detergent composition, may be added, for example poured, into the open pouches in the molds, and a second piece of fibrous structure may be placed over the active agents and/or detergent composition and in contact with the first piece of fibrous structure and the first piece of fibrous structure and second piece of fibrous structure are sealed together to form pouches, typically in such a manner as to at least partially enclose and/or completely enclose its internal volume and the active agents and/or detergent composition within its internal volume.

In another example, the pouch making process may be used to prepare pouches which have an internal volume that is divided into more than one compartment, typically known as a multi-compartment pouches. In the multi-compartment pouch process, the fibrous structure is folded at least twice, or at least three pieces of pouch wall materials (at least one of which is a fibrous pouch wall material, for example a water-soluble fibrous pouch wall material) are used, or at least two pieces of pouch wall materials (at least one of which is a fibrous pouch wall material, for example a water-soluble fibrous pouch wall material) are used wherein at least one piece of pouch wall material is folded at least once. The third piece of pouch wall material, when present, or a folded piece of pouch wall material, when present, creates a barrier layer that, when the pouch is sealed, divides the internal volume of said pouch into at least two compartments.

In another example, a process for making a multi-compartment pouch comprises fitting a first piece of the fibrous structure into a series of molds, for example the first piece of fibrous structure may be vacuum pulled into the molds so that the pouch wall material is flush with the inner walls of the molds. Active agents are typically poured into the open pouch formed by the first piece of fibrous structure in the molds. A pre-sealed compartment made of a pouch wall material can then be placed over the molds containing the composition. These pre-sealed compartments and said first piece of fibrous structure may be sealed together to form multi-compartment pouches, for example, dual-compartment pouches.

The pouches obtained from the processes of the present invention are water-soluble. The pouches are typically closed structures, made of a fibrous structure described herein, typically enclosing an internal volume which may comprise active agents and/or a detergent composition. The fibrous structures are suitable to hold active agents, e.g. without allowing the release of the active agents from the pouch prior to contact of the pouch with water. The exact execution of the pouch will depend on for example, the type and amount of the active agent in the pouch, the number of compartments in the pouch, the characteristics required from the pouch to hold, protect and deliver or release the active agents.

For multi-compartment pouches, the active agents and/or compositions contained in the different compartments may be the same or different. For example, incompatible ingredients may be contained in different compartments.

The pouches of the present invention may be of such a size that they conveniently contain either a unit dose amount of the active agents therein, suitable for the required operation, for example one wash, or only a partial dose, to allow the consumer greater flexibility to vary the amount used, for example depending on the size and/or degree of soiling of the wash load. The shape and size of the pouch is typically determined, at least to some extent, by the shape and size of the mold.

The multi-compartment pouches of the present invention may further be packaged in an outer package. Such an outer package may be a see-through or partially see-through container, for example a transparent or translucent bag, tub, carton or bottle. The pack can be made of plastic or any other suitable material, provided the material is strong enough to protect the pouches during transport. This kind of pack is also very useful because the user does not need to open the pack to see how many pouches remain in the package. Alternatively, the package may have non-see-through outer packaging, perhaps with indicia or artwork representing the visually-distinctive contents of the package.

### Non-Limiting Example for Making a Pouch

An example of a pouch of the present invention may be made as follows. Cut two layers of soluble fibrous structures according to the present invention at least twice the size of the pouch size intended to make. For example if finished pouch size has a planar footprint of about 2 inches×2 inches, then the pouch wall materials are cut 5 inches×5 inches. Next, lay both layers on top of one another on the heating element of an impulse sealer (Impulse Sealer model TISH-300 from TEW Electric Heating Equipment CO., LTD, 7F, No. 140, Sec. 2, Nan Kang Road, Taipei, Taiwan). The position of the layers on the heating element should be where a side closure seam is to be created. Close the sealer arm for 1 second to seal the two layers together. In a similar way, seal two more sides to create two additional side closure seams. With the three sides sealed, the two pouch wall materials form a pocket. Next, add the appropriate amount of powder into the pocket and then seal the last side to create the last side closure seam. A pouch is now formed. For most fibrous structures which are less than 0.2 mm thick, heating dial setting of 4 and heating time 1 second is used. Depending on the fibrous structures, heating temperature and heating time might have to be adjusted to realize a desirable seam. If the temperature is too low or the heating time is not long enough, the fibrous structure may not sufficiently melt and the two layers come apart easily; if the temperature is too high or the heating time is too long, pin holes may form at the sealed edge. One should adjust the sealing equipment conditions so as to the layers to melt and form a seam but not introduce negatives such as pin holes on the seam edge. Once the seamed pouch is formed, a scissor is used to trim off the excess material and leave a 1-2 mm edge on the outside of the seamed pouch.

### Methods of Use

The pouches of the present invention comprising one or more active agents, for example one or more fabric care active agents according the present invention may be utilized in a method for treating a fabric article. The method of treating a fabric article may comprise one or more steps selected from the group consisting of: (a) pre-treating the fabric article before washing the fabric article; (b) contacting the fabric article with a wash liquor formed by contacting the pouch with water; (c) contacting the fabric article with the pouch in a dryer; (d) drying the fabric article in the presence of the pouch in a dryer; and (e) combinations thereof.

In some embodiments, the method may further comprise the step of pre-moistening the pouch prior to contacting it to the fabric article to be pre-treated. For example, the pouch can be pre-moistened with water and then adhered to a portion of the fabric article comprising a stain that is to be pre-treated. Alternatively, the fabric article may be moistened and the pouch placed on or adhered thereto. In some embodiments, the method may further comprise the step of selecting of only a portion of the pouch for use in treating a fabric article. For example, if only one fabric care article is to be treated, a portion of the pouch may be cut and/or torn away and either placed on or adhered to the fabric article or placed into water to form a relatively small amount of wash liquor which is then used to pre-treat the fabric article. In this way, the user may customize the fabric treatment method according to the task at hand. In some embodiments, at least a portion of a pouch may be applied to the fabric article to be treated using a device. Exemplary devices include, but are not limited to, brushes, sponges and tapes. In yet another embodiment, the pouch may be applied directly to the surface of the fabric article. Any one or more of the aforementioned steps may be repeated to achieve the desired fabric treatment benefit for a fabric article.

### Comparative Example 1

A comparative fibrous element-forming composition according to Table 1, below, has been used to make comparative fibrous elements and ultimately a comparative soluble fibrous structure as described hereinabove in FIGS. 3 and 4. The Initial Water Propagation Rate, Hydration Value, Swelling Value, and Viscosity Value associated with the fibrous structure made from this fibrous element-forming composition are set forth in Table 10 below.

TABLE 1

| Raw Material | Formula (%) |
|---|---|
| Distilled Water | 79.00 |
| Fibrous element-forming material | 8.44 |
| (CMC, Ald C5678) | |
| Anionic Surfactant (Sodium | 5.11 |
| Laureth-1-Sulfate (SLE1S)) | |
| Anionic Surfactant (HSAS) | 0.81 |
| Nonionic Surfactant | 0.48 |
| Propanediol | 0.46 |
| Sodium Hydroxide | 0.29 |
| Anionic Surfactant (HLAS) | 3.00 |
| Fatty Acid (C12-18) | 0.20 |
| Builder (DTPA) | 0.45 |
| Suds Suppressor | 0.01 |
| Brightener | 0.06 |
| Rheology Modifier | 0.16 |
| (Polyacrylamide, NF221 PAM) | |
| Polyethyleneimine ethoxylate | 0.77 |
| Alkoxylated polyamine | 0.06 |
| Amine Oxide | 0.70 |
| TOTAL | 100.00 |

### Comparative Example 2

A comparative fibrous element-forming composition according to Table 2, below, is used to make comparative fibrous elements and ultimately a comparative soluble fibrous structure as described hereinabove in FIGS. 3 and 4. The Initial Water Propagation Rate, Hydration Value, Swelling Value, and Viscosity Value associated with this comparative soluble fibrous structure are set forth in Table 10 below.

TABLE 2

| Raw Material | Formula (%) |
|---|---|
| Distilled Water | 49.8 |
| Fibrous element-forming material | 5.4 |
| (CMC) | |
| Anionic Surfactant (Sodium | 18.7 |
| Laureth-1-Sulfate (SLE1S)) | |
| Anionic Surfactant (HSAS) | 1.6 |
| Nonionic Surfactant | 1.4 |
| Sodium Hydroxide | 1.8 |
| Anionic Surfactant (HLAS) | 9.7 |
| Fatty Acid | 6.0 |
| Builder (DTPA) | 1.6 |
| Suds Suppressor | 5.5 × 10⁻⁴ |
| Brightener | 3.4 × 10⁻³ |
| Rheology Modifier (Glycerol) | 4.0 |
| TOTAL | 100.0000 |

### Comparative Example 3

A comparative fibrous element-forming composition according to Table 3, below, is used to make comparative fibrous elements and ultimately a comparative soluble fibrous structure as described hereinabove in FIGS. 3 and 4. The Initial Water Propagation Rate, Hydration Value, Swelling Value, and Viscosity Value associated with this comparative soluble fibrous structure are set forth in Table 10 below.

TABLE 3

| Raw Material | Formula (%) |
|---|---|
| Distilled Water | 65.3300 |
| Fibrous element-forming material | 8.0700 |
| (Hydroxypropylmethylcellulose) | |
| Anionic surfactant Sodium | 20.8000 |
| Laureth-1-Sulfate (SLE1S) | |
| (Anionic surfactant) | |
| Amphoteric surfactant | 5.0000 |
| Citric Acid (Anhydrous) | 0.8000 |
| TOTAL | 100.0000 |

### Comparative Example 4

A comparative fibrous element-forming composition according to Table 4, below, is used to make comparative fibrous elements and ultimately a comparative soluble fibrous structure as described hereinabove in FIGS. 3 and 4. The Initial Water Propagation Rate, Hydration Value, Swelling Value, and Viscosity Value associated with this comparative soluble fibrous structure are set forth in Table 10 below.

TABLE 4

| Material | Formula (%) |
|---|---|
| Anionic Surfactant (High active NaAE3S) | 8.00 |
| Anionic Surfactant (HSAS) | 0.68 |
| Nonionic Surfactant | 0.87 |
| Sodium Hydroxide | 0.73 |
| Anionic Surfactant (C11.8 HLAS) | 4.00 |
| C12-18 Fatty Acid | 2.59 |
| Fibrous element-forming material (CMC) | 10.08 |
| Suds Suppressor | 0.06 |
| Polymeric Dispersant | 2.67 |
| Brightener | 0.07 |
| Chelant | 0.60 |
| Antimicrobial Agent | 0.01 |
| Rheology Modifier | 0.15 |
| Distilled Water | 68.65 |
| Diethylene Glycol | 0.84 |
| TOTAL | 100.01 |

### Inventive Example 1

A fibrous element-forming composition according to the present invention is set forth in Table 5 below is used to make fibrous elements and ultimately a soluble fibrous structure according to the present invention as described hereinabove in FIGS. 3 and 4. The Initial Water Propagation Rate, Hydration Value, Swelling Value, and Viscosity Value associated with this soluble fibrous structure are set forth in Table 10 below.

TABLE 5

| Raw Material | Formula (%) |
|---|---|
| Distilled Water | 60.0105 |
| Fibrous element-forming material (Polyvinylalcohol)¹ | 5.2750 |
| Fibrous element-forming material (Polyvinylalcohol)² | 5.2750 |
| Sodium Laureth-1-Sulfate (SLE1S) | 23.9455 |
| Amphoteric Surfactant | 5.2340 |
| Citric Acid (Anhydrous) | 0.2600 |
| TOTAL | 100.0000 |

¹PVA420H, M_{W} 75,000 g/mol, 78-82% hydrolyzed, available from Kuraray America, Inc.
²PVA403, M_{W} 30,000 g/mol, 78-82% hydrolyzed, available from Kuraray America, Inc.

### Inventive Example 2

A fibrous element-forming composition according to the present invention is set forth in Table 6 below is used to make fibrous elements and ultimately a soluble fibrous structure as described hereinabove in FIGS. 3 and 4. The Initial Water Propagation Rate, Hydration Value, Swelling Value, and Viscosity Value associated with this soluble fibrous structure are set forth in Table 10 below.

TABLE 6

| Raw Material | Formula (%) |
|---|---|
| Distilled Water | 59.4001 |
| Tri Quat | 0.0960 |
| Cationic Guar Polymer | 0.5144 |
| Fibrous element-forming material (Polyvinylalcohol)¹ | 5.2750 |
| Fibrous element-forming material (Polyvinylalcohol)² | 5.2750 |
| Anionic Surfactant (Sodium Laureth-1-Sulfate (SLE1S)) | 23.9455 |
| Anionic Surfactant (Sodium Laureth-3-Sulfate (SLE3S)) | 0.0000 |
| Amphoteric Surfactant | 5.2340 |
| Citric Acid (Anhydrous) | 0.2600 |
| Total | 100.0000 |

¹PVA420H, M_{W} 75,000 g/mol, 78-82% hydrolyzed, available from Kuraray America, Inc.
²PVA403, M_{W} 30,000 g/mol, 78-82% hydrolyzed, available from Kuraray America, Inc.

### Inventive Example 3

A fibrous element-forming composition according to the present invention is set forth in Table 7 below is used to make fibrous elements and ultimately a soluble fibrous structure as described hereinabove in FIGS. 3 and 4. The Initial Water Propagation Rate, Hydration Value, Swelling Value, and Viscosity Value associated with this soluble fibrous structure are set forth in Table 10 below.

TABLE 7

| Raw Material | Formula (%) |
|---|---|
| Distilled Water | 71.2500 |
| Fibrous element-forming material (Carboxymethylcellulose) | 14.3000 |
| Nonionic surfactant (Alkyl polyglucoside - The Dow | 14.3000 |
| Chemical Company) | |
| Rheology Modifier (Polyacrylamide - SNF, Inc.) | 0.1500 |
| TOTAL | 100.0000 |

### Inventive Example 4

A fibrous element-forming composition according to the present invention is set forth in Table 8 below is used to make fibrous elements and ultimately a soluble fibrous structure as described hereinabove in FIGS. 3 and 4. The Initial Water Propagation Rate, Hydration Value, Swelling Value, and Viscosity Value associated with this soluble fibrous structure are set forth in Table 10 below.

TABLE 8

| Raw Material | Formula (%) |
|---|---|
| Distilled Water | 59.9539 |
| Fibrous element-forming material (Polyvinylalcohol)¹ | 3.7685 |
| Fibrous element-forming material (Polyvinylalcohol)² | 8.9028 |
| Anionic Surfactant (Sodium Laureth-1-Sulfate (SLE1S)) | 20.4000 |
| Cocofatty Acid Monoethanol Amide | 3.7230 |
| Amphoteric Surfactant | 3.0100 |
| Citric Acid (Anhydrous) | 0.2418 |
| TOTAL | 100.0000 |

¹PVA420H, M_{W} 75,000 g/mol, 78-82% hydrolyzed, available from Kuraray America, Inc.
²PVA403, M_{W} 30,000 g/mol, 78-82% hydrolyzed, available from Kuraray America, Inc.

### Inventive Example 5

A fibrous element-forming composition according to the present invention is set forth in Table 9 below is used to make fibrous elements and ultimately a soluble fibrous structure as described hereinabove in FIGS. 3 and 4. The Initial Water Propagation Rate, Hydration Value, Swelling Value, and Viscosity Value associated with this soluble fibrous structure are set forth in Table 10 below.

TABLE 9

| Raw Material | Formula (%) |
|---|---|
| Distilled Water | 59.5950 |
| Fibrous element-forming material (Polyvinylalcohol)¹ | 3.7600 |
| Fibrous element-forming material (Polyvinylalcohol)² | 8.9000 |
| Cationic Guar Polymer | 0.4000 |
| Cocofatty Acid Monoethanol Amide | 3.7200 |
| Amphoteric Surfactant | 3.0100 |
| Anionic Surfactant (Sodium Laureth-1-Sulfate (SLE1S)) | 7.6540 |
| Anionic Surfactant (Sodium Laureth-3-Sulfate (SLE3S)) | 2.2510 |
| Anionic Surfactant (Sodium Undecyl Sulfate) | 10.4500 |
| Citric Acid (Anhydrous) | 0.2600 |
| TOTAL | 100.0000 |

¹PVA420H, M_{W} 75,000 g/mol, 78-82% hydrolyzed, available from Kuraray America, Inc.
²PVA403, M_{W} 30,000 g/mol, 78-82% hydrolyzed, available from Kuraray America, Inc.

### Soluble Fibrous Structure Properties Table

TABLE 10

| | | | | Vis- | Dissolu- |
|---|---|---|---|---|---|
| Soluble | Initial Water | Hydration | Swell- | cosity | tion |
| Fibrous | Propagation | Value | ing | Value | Time |
| Structure | Rate (m/s) | (m/s^{1/2}) | Value | (Pa · s) | (s) |
| Comparative | 2.08 × 10⁻⁴ | 7.60 × 10⁻⁵ | 2.13 | 270.46 | 690 |
| Example 1 | | | | | |
| Comparative | 3.45 × 10⁻⁴ | 6.75 × 10⁻⁵ | 2.16 | 796.86 | 550 |
| Example 2 | | | | | |
| Comparative | 4.79 × 10⁻⁴ | 5.94 × 10⁻⁵ | 2.59 | 101.00 | 320 |
| Example 3 | | | | | |
| Comparative | 4.26 × 10⁻⁴ | 4.36 × 10⁻⁵ | 2.88 | 196.00 | 705 |
| Example 4 | | | | | |
| Inventive | 2.89 × 10⁻³ | 9.75 × 10⁻⁵ | 1.41 | 5.96 | 34 |
| Example 1 | | | | | |
| Inventive | 2.50 × 10⁻³ | — | — | 3.84 | 25 |
| Example 2 | | | | | |
| Inventive | 6.71 × 10⁻³ | — | — | 1.05 | 20 |
| Example 3 | | | | | |
| Inventive | 4.51 × 10⁻² | 1.88 × 10⁻⁴ | 1.47 | 1.46 | 1.58 |
| Example 4 | | | | | |
| Inventive | 2.53 × 10⁻³ | 1.33 × 10⁻⁴ | 1.82 | 1.00 | 1.37 |
| Example 5 | | | | | |

### Test Methods

Unless otherwise indicated, all tests described herein including those described under the Definitions section and the following test methods are conducted on samples that have been conditioned in a conditioned room at a temperature of 23° C.±1° C. and a relative humidity of 50%±2% for 2 hours prior to the test unless otherwise indicated. Samples conditioned as described herein are considered dry samples (such as “dry fibrous elements”) for purposes of this invention. Further, all tests are conducted in such conditioned room.

### Water Content Test Method

The water (moisture) content present in a filament and/or fiber and/or soluble fibrous structure is measured using the following Water Content Test Method.

A filament and/or soluble fibrous structure or portion thereof (“sample”) is placed in a conditioned room at a temperature of 23° C.±1° C. and a relative humidity of 50%±2% for at least 24 hours prior to testing. The weight of the sample is recorded when no further weight change is detected for at least a 5 minute period. Record this weight as the “equilibrium weight” of the sample. Next, place the sample in a drying oven for 24 hours at 70° C. with a relative humidity of about 4% to dry the sample. After the 24 hours of drying, immediately weigh the sample. Record this weight as the “dry weight” of the sample. The water (moisture) content of the sample is calculated as follows:

$$\% Water ( moisture ) in sample = 100 \% \times \frac{( Equilibrium weight of sample - Dry weight of sample )}{Dry weight of sample}$$

The % Water (moisture) in sample for 3 replicates is averaged to give the reported % Water (moisture) in sample.

### Dissolution Test Method

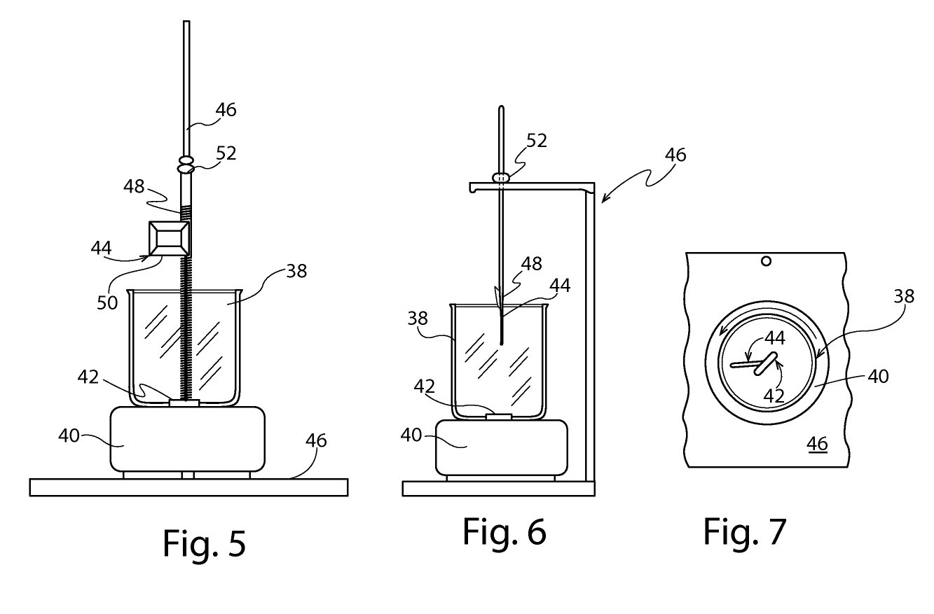

### Apparatus and Materials (FIGS. 5 Through 7):

600 mL Beaker 38

Magnetic Stirrer 40 (Labline Model No. 1250 or equivalent)

Magnetic Stirring Rod 42 (5 cm)

Thermometer (1 to 100° C.+/−1° C.)

Cutting Die—Stainless Steel cutting die with dimensions 3.8 cm×3.2 cm

Timer (0-3,600 seconds or 1 hour), accurate to the nearest second. Timer used should have sufficient total time measurement range if sample exhibits dissolution time greater than 3,600 seconds. However, timer needs to be accurate to the nearest second.

Polaroid 35 mm Slide Mount 44 (commercially available from Polaroid Corporation or equivalent)

35 mm Slide Mount Holder 46 (or equivalent)

City of Cincinnati Water or equivalent having the following properties: Total Hardness=155 mg/L as CaCO₃; Calcium content=33.2 mg/L; Magnesium content=17.5 mg/L; Phosphate content=0.0462.

### Test Protocol

Equilibrate samples in constant temperature and humidity environment of 23° C.±1° C. and 50% RH±2% for at least 2 hours.

Measure the basis weight of the sample materials using Basis Weight Method defined herein.

Cut three dissolution test specimens from soluble fibrous structure sample using cutting die (3.8 cm×3.2 cm), so it fits within the 35 mm slide mount 44 which has an open area dimensions 24×36 mm.

Lock each specimen in a separate 35 mm slide mount 44.

Place magnetic stirring rod 42 into the 600 mL beaker 38.

Turn on the city water tap flow (or equivalent) and measure water temperature with thermometer and, if necessary, adjust the hot or cold water to maintain it at the testing temperature. Testing temperature is 15° C.±1° C. water. Once at testing temperature, fill beaker 240 with 500 mL±5 mL of the 15° C.±1° C. city water.

Place full beaker 38 on magnetic stirrer 40, turn on stirrer 40, and adjust stir speed until a vortex develops and the bottom of the vortex is at the 400 mL mark on the beaker 38.

Secure the 35 mm slide mount 44 in the alligator clamp 48 of the 35 mm slide mount holder 46 such that the long end 50 of the slide mount 44 is parallel to the water surface. The alligator clamp 48 should be positioned in the middle of the long end 50 of the slide mount 44. The depth adjuster 52 of the holder 46 should be set so that the distance between the bottom of the depth adjuster 52 and the bottom of the alligator clamp 48 is 11±0.125 inches. This set up will position the sample surface perpendicular to the flow of the water. A slightly modified example of an arrangement of a 35 mm slide mount and slide mount holder are shown in FIGS. 1-3 of U.S. Pat. No. 6,787,512.

In one motion, drop the secured slide and clamp into the water and start the timer. The sample is dropped so that the sample is centered in the beaker. Disintegration occurs when the soluble fibrous structure breaks apart. Record this as the disintegration time. When all of the visible soluble fibrous structure is released from the slide mount, raise the slide out of the water while continuing the monitor the solution for undissolved soluble fibrous structure fragments. Dissolution occurs when all soluble fibrous structure fragments are no longer visible. Record this as the dissolution time.

Three replicates of each sample are run and the average disintegration and dissolution times are recorded. Average disintegration and dissolution times are in units of seconds.

The average disintegration and dissolution times are normalized for basis weight by dividing each by the sample basis weight as determined by the Basis Weight Method defined herein. Basis weight normalized disintegration and dissolution times are in units of seconds/gsm of sample (s/(g/m²)).

### Diameter Test Method

The diameter of a discrete fibrous element or a fibrous element within a soluble fibrous structure or film is determined by using a Scanning Electron Microscope (SEM) or an Optical Microscope and an image analysis software. A magnification of 200 to 10,000 times is chosen such that the fibrous elements are suitably enlarged for measurement. When using the SEM, the samples are sputtered with gold or a palladium compound to avoid electric charging and vibrations of the fibrous element in the electron beam. A manual procedure for determining the fibrous element diameters is used from the image (on monitor screen) taken with the SEM or the optical microscope. Using a mouse and a cursor tool, the edge of a randomly selected fibrous element is sought and then measured across its width (i.e., perpendicular to fibrous element direction at that point) to the other edge of the fibrous element. A scaled and calibrated image analysis tool provides the scaling to get actual reading in p.m. For fibrous elements within a soluble fibrous structure or film, several fibrous element are randomly selected across the sample of the soluble fibrous structure or film using the SEM or the optical microscope. At least two portions the soluble fibrous structure or film (or web inside a product) are cut and tested in this manner. Altogether at least 100 such measurements are made and then all data are recorded for statistical analysis. The recorded data are used to calculate average (mean) of the fibrous element diameters, standard deviation of the fibrous element diameters, and median of the fibrous element diameters.

Another useful statistic is the calculation of the amount of the population of fibrous elements that is below a certain upper limit. To determine this statistic, the software is programmed to count how many results of the fibrous element diameters are below an upper limit and that count (divided by total number of data and multiplied by 100%) is reported in percent as percent below the upper limit, such as percent below 1 micrometer diameter or %−submicron, for example. We denote the measured diameter (in μm) of an individual circular fibrous element as di.

In case the fibrous elements have non-circular cross-sections, the measurement of the fibrous element diameter is determined as and set equal to the hydraulic diameter which is four times the cross-sectional area of the fibrous element divided by the perimeter of the cross-section of the fibrous element (outer perimeter in case of hollow fibrous elements). The number-average diameter, alternatively average diameter is calculated as:

$${d}_{num} = \frac{{\sum }_{i = 1}^{n} {d}_{i}}{n}$$

### Thickness Method

Thickness of a soluble fibrous structure or film is measured by cutting 5 samples of a soluble fibrous structure or film sample such that each cut sample is larger in size than a load foot loading surface of a VIR Electronic Thickness Tester Model II available from Thwing-Albert Instrument Company, Philadelphia, Pa. Typically, the load foot loading surface has a circular surface area of about 3.14 in². The sample is confined between a horizontal flat surface and the load foot loading surface. The load foot loading surface applies a confining pressure to the sample of 15.5 g/cm². The caliper of each sample is the resulting gap between the flat surface and the load foot loading surface. The caliper is calculated as the average caliper of the five samples. The result is reported in millimeters (mm).

### Basis Weight Test Method

Basis weight of a fibrous structure sample is measured by selecting twelve (12) individual fibrous structure samples and making two stacks of six individual samples each. If the individual samples are connected to one another vie perforation lines, the perforation lines must be aligned on the same side when stacking the individual samples. A precision cutter is used to cut each stack into exactly 3.5 in.×3.5 in. squares. The two stacks of cut squares are combined to make a basis weight pad of twelve squares thick. The basis weight pad is then weighed on a top loading balance with a minimum resolution of 0.01 g. The top loading balance must be protected from air drafts and other disturbances using a draft shield. Weights are recorded when the readings on the top loading balance become constant. The Basis Weight is calculated as follows:

$$Basis Weight ( lbs \text{/} 3000 {ft}^{2} ) = \frac{Weight of basis weight pad ( g ) \times 3000 {ft}^{2}}{\begin{matrix}453.6 g \text{/} lbs \times 12 samples \times \\ [ 12.25 {in}^{2} ( Area of basis weight pad ) / 144 {in}^{2} ]\end{matrix}}$$

$$Basis Weight ( g \text{/} {m}^{2} ) = \frac{Weight of basis weight pad ( g ) \times 10 , 000 {cm}^{2} \text{/} {m}^{2}}{79.0321 {cm}^{2} ( Area of basis weight pad ) \times 12 samples}$$

If fibrous structure sample is smaller than 3.5 in.×3.5 in., then smaller sampling areas can be used for basis weight determination with associated changes to the calculations.

### Weight Average Molecular Weight Test Method

The weight average molecular weight (Mw) of a material, such as a polymer, is determined by Gel Permeation Chromatography (GPC) using a mixed bed column. A high performance liquid chromatograph (HPLC) having the following components: Millenium®, Model 600E pump, system controller and controller software Version 3.2, Model 717 Plus autosampler and CHM-009246 column heater, all manufactured by Waters Corporation of Milford, Mass., USA, is utilized. The column is a PL gel 20 μm Mixed A column (gel molecular weight ranges from 1,000 g/mol to 40,000,000 g/mol) having a length of 600 mm and an internal diameter of 7.5 mm and the guard column is a PL gel 20 μm, 50 mm length, 7.5 mm ID. The column temperature is 55° C. and the injection volume is 200 μL. The detector is a DAWN® Enhanced Optical System (EOS) including Astra® software, Version 4.73.04 detector software, manufactured by Wyatt Technology of Santa Barbara, Calif., USA, laser-light scattering detector with K5 cell and 690 nm laser. Gain on odd numbered detectors set at 101. Gain on even numbered detectors set to 20.9. Wyatt Technology's Optilab® differential refractometer set at 50° C. Gain set at 10. The mobile phase is HPLC grade dimethylsulfoxide with 0.1% w/v LiBr and the mobile phase flow rate is 1 mL/min, isocratic. The run time is 30 minutes.

A sample is prepared by dissolving the material in the mobile phase at nominally 3 mg of material/1 mL of mobile phase. The sample is capped and then stirred for about 5 minutes using a magnetic stirrer. The sample is then placed in an 85° C. convection oven for 60 minutes. The sample is then allowed to cool undisturbed to room temperature. The sample is then filtered through a 5 μm Nylon membrane, type Spartan-25, manufactured by Schleicher & Schuell, of Keene, N.H., USA, into a 5 milliliter (mL) autosampler vial using a 5 mL syringe.

For each series of samples measured (3 or more samples of a material), a blank sample of solvent is injected onto the column. Then a check sample is prepared in a manner similar to that related to the samples described above. The check sample comprises 2 mg/mL of pullulan (Polymer Laboratories) having a weight average molecular weight of 47,300 g/mol. The check sample is analyzed prior to analyzing each set of samples. Tests on the blank sample, check sample, and material test samples are run in duplicate. The final run is a run of the blank sample. The light scattering detector and differential refractometer is run in accordance with the “Dawn EOS Light Scattering Instrument Hardware Manual” and “Optilab® DSP Interferometric Refractometer Hardware Manual,” both manufactured by Wyatt Technology Corp., of Santa Barbara, Calif., USA, and both incorporated herein by reference.

The weight average molecular weight of the sample is calculated using the detector software. A dn/dc (differential change of refractive index with concentration) value of 0.066 is used. The baselines for laser light detectors and the refractive index detector are corrected to remove the contributions from the detector dark current and solvent scattering. If a laser light detector signal is saturated or shows excessive noise, it is not used in the calculation of the molecular mass. The regions for the molecular weight characterization are selected such that both the signals for the 90° detector for the laser-light scattering and refractive index are greater than 3 times their respective baseline noise levels. Typically the high molecular weight side of the chromatogram is limited by the refractive index signal and the low molecular weight side is limited by the laser light signal.

The weight average molecular weight can be calculated using a “first order Zimm plot” as defined in the detector software. If the weight average molecular weight of the sample is greater than 1,000,000 g/mol, both the first and second order Zimm plots are calculated, and the result with the least error from a regression fit is used to calculate the molecular mass. The reported weight average molecular weight is the average of the two runs of the material test sample.

### Tensile Test Method: Elongation, Tensile Strength, TEA and Modulus

Elongation, Tensile Strength, TEA, Secant Modulus and Tangent Modulus are measured on a constant rate of extension tensile tester with computer interface (a suitable instrument is the MTS Insight using Testworks 4.0 Software, as available from MTS Systems Corp., Eden Prairie, Minn.) using a load cell for which the forces measured are within 10% to 90% of the limit of the cell. Both the movable (upper) and stationary (lower) pneumatic jaws are fitted with rubber faced grips, 25.4 mm in height and wider than the width of the test specimen. An air pressure of about 80 psi is supplied to the jaws. All testing is performed in a conditioned room maintained at about 23° C.±1 C.° and about 50%±2% relative humidity. Samples are conditioned under the same conditions for 2 hours before testing.

Eight specimens of soluble fibrous structure and/or dissolving fibrous structure are divided into two stacks of four specimens each. The specimens in each stack are consistently oriented with respect to machine direction (MD) and cross direction (CD). One of the stacks is designated for testing in the MD and the other for CD. Using a one inch precision cutter (Thwing Albert JDC-1-10, or similar) cut four MD strips from one stack, and four CD strips from the other, with dimensions of 2.54 cm±0.02 cm wide by at least 50 mm long.

Program the tensile tester to perform an extension test, collecting force and extension data at an acquisition rate of 100 Hz. Initially lower the crosshead 6 mm at a rate of 5.08 cm/min to introduce slack in the specimen, then raise the crosshead at a rate of 5.08 cm/min until the specimen breaks. The break sensitivity is set to 80%, i.e., the test is terminated when the measured force drops to 20% of the maximum peak force, after which the crosshead is returned to its original position.

Set the gage length to 2.54 cm. Zero the crosshead. Insert a specimen into the upper grip, aligning it vertically within the upper and lower jaws and close the upper grips. With the sample hanging from the top grips, zero the load cell. Insert the specimen into the lower grips and close. With the grips closed the specimen should be under enough tension to eliminate any slack but exhibits a force less than 3.0 g on the load cell. Start the tensile tester and data collection. Repeat testing in like fashion for all four CD and four MD specimens.

Program the software to calculate the following from the constructed force (g) verses extension (cm) curve:

Tensile Strength is the maximum peak force (g) divided by the specimen width (cm) and reported as g/cm to the nearest 1.0 g/cm.

Adjusted Gage Length is calculated as the extension measured at 3.0 g of force (cm) added to the original gage length (cm).

Elongation is calculated as the extension at maximum peak force (cm) divided by the Adjusted Gage Length (cm) multiplied by 100 and reported as % to the nearest 0.1%

Total Energy (TEA) is calculated as the area under the force curve integrated from zero extension to the extension at the maximum peak force (g*cm), divided by the product of the adjusted Gage Length (cm) and specimen width (cm) and is reported out to the nearest 1 g*cm/cm².

Replot the force (g) verses extension (cm) curve as a force (g) verses strain (%) curve. Strain is herein defined as the extension (cm) divided by the Adjusted Gage Length (cm)×100. Program the software to calculate the following from the constructed force (g) verses strain (%) curve:

The Secant Modulus is calculated from a least squares linear fit of the steepest slope of the force vs strain curve using a cord that has a rise of at least 20% of the peak force. This slope is then divided by the specimen width (2.54 cm) and reported to the nearest 1.0 g/cm.

Tangent Modulus is calculated as the slope the line drawn between the two data points on the force (g) versus strain (%) curve. The first data point used is the point recorded at 28 g force, and the second data point used is the point recorded at 48 g force. This slope is then divided by the specimen width (2.54 cm) and reported to the nearest 1.0 g/cm.

The Tensile Strength (g/cm), Elongation (%), Total Energy (g*cm/cm²), Secant Modulus (g/cm) and Tangent Modulus (g/cm) are calculated for the four CD specimens and the four MD specimens. Calculate an average for each parameter separately for the CD and MD specimens.

### Calculations:

Total Dry Tensile Strength (TDT)=MD Tensile Strength (g/cm)+CD Tensile Strength (g/cm)

Geometric Mean Tensile=Square Root of [MD Tensile Strength (g/cm)×CD Tensile Strength (g/cm)]

Tensile Ratio=MD Tensile Strength (g/cm)/CD Tensile Strength (g/cm)

Geometric Mean Peak Elongation=Square Root of [MD Elongation (%)×CD Elongation (%)]

Total TEA=MD TEA (g*cm/cm²)+CD TEA (g*cm/cm²)

Geometric Mean TEA=Square Root of [MD TEA (g*cm/cm²)×CD TEA (g*cm/cm²)]

Geometric Mean Tangent Modulus=Square Root of [MD Tangent Modulus (g/cm)×CD Tangent Modulus (g/cm)]

Total Tangent Modulus=MD Tangent Modulus (g/cm)+CD Tangent Modulus (g/cm)

Geometric Mean Secant Modulus=Square Root of [MD Secant Modulus (g/cm)×CD Secant Modulus (g/cm)]

Total Secant Modulus=MD Secant Modulus (g/cm)+CD Secant Modulus (g/cm)

### Plate Stiffness Test Method

As used herein, the “Plate Stiffness” test is a measure of stiffness of a flat sample as it is deformed downward into a hole beneath the sample. For the test, the sample is modeled as an infinite plate with thickness “t” that resides on a flat surface where it is centered over a hole with radius “R”. A central force “F” applied to the tissue directly over the center of the hole deflects the tissue down into the hole by a distance “w”. For a linear elastic material the deflection can be predicted by:

$$w = \frac{3 F}{4 π {Et}^{3}} ( 1 - v ) ( 3 + v ) {R}^{2}$$

where “E” is the effective linear elastic modulus, “v” is the Poisson's ratio, “R” is the radius of the hole, and “t” is the thickness of the tissue, taken as the caliper in millimeters measured on a stack of 5 tissues under a load of about 0.29 psi. Taking Poisson's ratio as 0.1 (the solution is not highly sensitive to this parameter, so the inaccuracy due to the assumed value is likely to be minor), the previous equation can be rewritten for “w” to estimate the effective modulus as a function of the flexibility test results:

$$E \approx \frac{3 {R}^{2}}{4 {t}^{3}} \frac{F}{w}$$

The test results are carried out using an MTS Alliance RT/1 testing machine (MTS Systems Corp., Eden Prairie, Minn.) with a 100N load cell. As a stack of five tissue sheets at least 2.5-inches square sits centered over a hole of radius 15.75 mm on a support plate, a blunt probe of 3.15 mm radius descends at a speed of 20 mm/min. When the probe tip descends to 1 mm below the plane of the support plate, the test is terminated. The maximum slope in grams of force/mm over any 0.5 mm span during the test is recorded (this maximum slope generally occurs at the end of the stroke). The load cell monitors the applied force and the position of the probe tip relative to the plane of the support plate is also monitored. The peak load is recorded, and “E” is estimated using the above equation.

The Plate Stiffness “S” per unit width can then be calculated as:

$$S = \frac{{Et}^{3}}{12}$$

and is expressed in units of Newtons-millimeters. The Testworks program uses the following formula to calculate stiffness:

S=(F/w)[(3+v)R²/16π]

wherein “F/w” is max slope (force divided by deflection), “v” is Poisson's ratio taken as 0.1, and “R” is the ring radius.

### Fibrous Element Composition Test Method

In order to prepare fibrous elements for fibrous element composition measurement, the fibrous elements must be conditioned by removing any coating compositions and/or materials present on the external surfaces of the fibrous elements that are removable. A chemical analysis of the conditioned fibrous elements is then completed to determine the compositional make-up of the fibrous elements with respect to the fibrous element-forming materials and the active agents and the level of the fibrous element-forming materials and active agents present in the fibrous elements.

The compositional make-up of the fibrous elements with respect to the fibrous element-forming material and the active agents can also be determined by completing a cross-section analysis using TOF-SIMs or SEM. Still another method for determining compositional make-up of the fibrous elements uses a fluorescent dye as a marker. In addition, as always, a manufacturer of fibrous elements should know the compositions of their fibrous elements.

### Median Particle Size Test Method

This test method must be used to determine median particle size.

The median particle size test is conducted to determine the median particle size of the seed material using ASTM D 502-89, “Standard Test Method for Particle Size of Soaps and Other Detergents”, approved May 26, 1989, with a further specification for sieve sizes used in the analysis. Following section 7, “Procedure using machine-sieving method,” a nest of clean dry sieves containing U.S. Standard (ASTM E 11) sieves #8 (2360 um), #12 (1700 um), #16 (1180 um), #20 (850 um), #30 (600 um), #40 (425 um), #50 (300 um), #70 (212 um), #100 (150 um) is required. The prescribed Machine-Sieving Method is used with the above sieve nest. The seed material is used as the sample. A suitable sieve-shaking machine can be obtained from W.S. Tyler Company of Mentor, Ohio, U.S.A.

The data are plotted on a semi-log plot with the micron size opening of each sieve plotted against the logarithmic abscissa and the cumulative mass percent (Q₃) plotted against the linear ordinate. An example of the above data representation is given in ISO 9276-1:1998, “Representation of results of particle size analysis—Part 1: Graphical Representation”, Figure A.4. The seed material median particle size (D₅₀), for the purpose of this invention, is defined as the abscissa value at the point where the cumulative mass percent is equal to 50 percent, and is calculated by a straight line interpolation between the data points directly above (a50) and below (b50) the 50% value using the following equation:

D₅₀=10̂[Log(Dₐ₅₀)−(Log(Dₐ₅₀)−Log(D_{b50}))*(Qₐ₅₀−50%)/(Qₐ₅₀−Q_{b50})]

where Qₐ₅₀ and Q_{b50} are the cumulative mass percentile values of the data immediately above and below the 50^{th} percentile, respectively; and Dₐ₅₀ and D_{b50} are the micron sieve size values corresponding to these data.

In the event that the 50^{th} percentile value falls below the finest sieve size (150 um) or above the coarsest sieve size (2360 um), then additional sieves must be added to the nest following a geometric progression of not greater than 1.5, until the median falls between two measured sieve sizes.

The Distribution Span of the Seed Material is a measure of the breadth of the seed size distribution about the median. It is calculated according to the following:

Span=(D₈₄/D₅₀+D₅₀/D₁₆)/2
Where D₅₀ is the median particle size and D₈₄ and D₁₆ are the particle sizes at the sixteenth and eighty-fourth percentiles on the cumulative mass percent retained plot, respectively.

In the event that the D₁₆ value falls below the finest sieve size (150 um), then the span is calculated according to the following:

Span=(D₈₄/D₅₀).

In the event that the D₈₄ value falls above the coarsest sieve size (2360 um), then the span is calculated according to the following:

Span=(D₅₀/D₁₆).

In the event that the D₁₆ value falls below the finest sieve size (150 um) and the D₈₄ value falls above the coarsest sieve size (2360 um), then the distribution span is taken to be a maximum value of 5.7.

### Additional Soluble Fibrous Structure Test Methods

The following test methods (Initial Water Propagation Rate, Hydration Value, Swelling Value, and Viscosity Value) are conducted on samples that have been conditioned at a temperature of 23° C.±2.0° C. and a relative humidity of 45%±10% for a minimum of 12 hours prior to the test. Except where noted all tests are conducted in such a conditioned room, and all tests are conducted under the same environmental conditions. Any damaged product is discarded. Samples that have defects such as wrinkles, tears, holes, and alike are not tested. All instruments are calibrated according to manufacturer's specifications. Samples conditioned as described herein are considered dry samples for purposes of this invention. At least three samples are measured for any given material being tested, and the results from those three or more replicates are averaged to give the final reported value for that material in that test. When conducting single fibrous element tests on materials comprising more than one type of fibrous element (as distinguished by fibrous element size, shape, colour, density, crystallinity, chemical composition, or other discernible characteristic), at least three replicate samples are tested for each type of fibrous element, and the results reported as the average for each type of fibrous element.

### Initial Water Propagation Rate Test Method

One of skill understands that obtaining a suitable sample from a fibrous article may involve several preparation steps, which may include the removal of lotions or fluids coating the article and/or fibrous material, or the separation of the various components from each other and from other components of the finished article. Furthermore, one of skill understands it is important to ensure that preparation steps for testing a fibrous sample do not damage the sample to be tested or alter the characteristics to be measured. A clean dry fibrous sample is the intended starting point for the measurement.

The Initial Water Propagation Rate (ν(0)) is determined by testing a sample of the fibrous structure, for example soluble fibrous structure, fabric, or nonwoven material. The test is conducted using an upright compound light microscope, such as a Nikon Eclipse LV100POL (Nikon Instruments Inc., Melville, N.Y., U.S.A.) or equivalent. The microscope is equipped with long working distance, flat-field corrected objective lenses of 10× or 20× magnification, such as Nikon CF Plan EPI ELWD (Nikon Instruments Inc., Melville, N.Y., U.S.A.) or equivalent. The microscope is also equipped with a high-speed video camera capable of capturing at least 200 frames s⁻¹ for 12.5 seconds, with at least 1024×512 pixels per frame, while capturing images having a minimum spatial resolution of 1.5 μm per pixel or higher resolution (i.e., a higher resolution corresponds to less distance per pixel). Suitable cameras include the Phantom V310 (Vision Research Inc., Wayne, N.J., U.S.A.) or equivalent. The microscope is aligned for Koehler Illumination and spatial measurements in the x-y image plane are calibrated using a stage micrometer. Samples are imaged and measured in either brightfield transmission mode or brightfield epi-illumination mode. Computer software programs may be used to control the video camera and to assist in the capture and spatial measurement analysis of images. Suitable software programs include Image-Pro Premier 64-bit, version 9.0.4, (Media Cybernetics Inc., Rockville, Md., U.S.A.) or equivalent).

Test samples are prepared by cutting the dry fibrous material, soluble fibrous structure, web, or nonwoven to be tested in order to obtain a 5 mm×10 mm rectangular shaped sample piece. A new sharp razor blade is used to cut each sample and care is taken to not compress the edges of the sample. The sample is laid down flat across a standard 25 mm×75 mm glass microscope slide such that the long axis of the sample is perpendicular to the long axis of the glass slide.

The sample is observed under the microscope using brightfield transmission mode illumination. If light is observed to pass through the sample then images of the sample are obtained using brightfield transmission mode illumination. If light does not appear to pass through the sample when observed in transmission mode, then images of the sample are obtained using brightfield epi-illumination mode.

A shallow flow channel with water-impermeable side walls is created running across the microscope slide, with the sample centered across both the width and length of the channel. The channel is 6 to 7 mm in width and 15 to 25 mm in length. The sides of the channel can be created from pressure-sensitive adhesive office tape, such as invisible Scotch Magic Office Tape (3M Company, Saint Paul, Minn., U.S.A.), by firmly placing strips of tape onto the glass slide so that each strip is adjacent and parallel to a long side of the sample. The tape will be very close to the sample but not touch the sample. The sides of the channel are made higher by repeatedly placing additional layers of tape on top of the previous layers. The final height of the two side walls of the channel is approximately 0.5 mm greater than the thickness of the web sample. A glass cover slip (thickness number 1.5) is placed on top of both side walls of the channel so that it bridges across the channel to form a ceiling above the sample. The cover slip is secured into place with adhesive tape such that it allows for unobstructed microscopic observation of the sample through the cover slip. The slide with channel-mounted sample is placed onto the microscope stage, the sample is brought into focus and positioned such that an image captured by the video camera is mostly filled with sample material. Additionally the sample is positioned such that the long axis of the image is parallel to the long axis of the sample, and a short-side edge of the web can be clearly observed within the captured image.

The capture of time-stamped photomicrograph video images of the positioned sample is commenced at the same time that laboratory-grade filtered deionized (DI) water begins being dispensed very slowly into the channel from a 1 mL syringe filled with 23° C.±2° C. DI water. The DI water is dispensed between the slide and the cover slip into the open end of the channel which is closest to the sample edge being imaged. Care is taken to ensure that the volume and pressure of water dispensed are both sufficiently low that a water front is created which advances up the channel and touches the nearest short-edge of the web gently and is then drawn into the web by capillary and wicking forces, but is not forced into the web under pressure nor floods under the web such that the sample is floated or moves. After making initial contact at the web's short edge, the water front advances through the length of the web sample. The movement of the water front and its penetration within the web is captured in the photomicrograph video images, and the distance travelled by the front is measured over time. The propagating water front is defined as the vertical water-air interface advancing laterally within the web at a given time point, as observed visually in the photomicrograph images. Determination of the position of the propagating water front may be facilitated by noting the visual change in opacity or whiteness of the web which occurs as the material is wetted. The capture of video images is continued until one of the following conditions is met, namely: the water front has penetrated throughout the whole of the sample observed within the field of view, or a time period of 12.5 seconds has been captured. The change in the location of the water front within the web is measured as the distance travelled over time and is used to calculate the rate at which water propagates through the web over time.

Linear spatial measurements along the length of the sample are made from a time series of images which are a subset of the image frames in a captured video. Each time series covers the timespan from when the advancing water front is first observed contacting the edge of the web, through until when the water front has propagated throughout the whole of the web sample within the field of view. To create a time series of images from a captured video, the frame of the video in which the advancing water front is first observed coming into contact with the edge of the web is identified and recorded as the first frame of the time series. The time stamp value recorded at the time of capture for this first image in the time series is defined as time zero (t=0) for that time series, and is recorded. The time series is then extended by adding additional frames from the same video, progressing from the time zero image to the subsequent images in the order in which they were captured. For a given image captured after time zero, the elapsed time (t) in seconds is defined as the absolute difference in time between time zero for that time series and the time of capture for the given image. These additional images are selected such that their times of capture are temporally spaced apart by intervals of approximately 0.05 seconds. This process of adding images to the time series is continued until an image is added whose time of capture is at least 1 second after time zero. After this 1 second of elapsed time is reached, additional images are then selected from the video at a temporal spacing of 0.5 second intervals and these images continue to be added until the time series spans the period from time zero through until one of the following conditions is met, namely: the water front has propagated across the entire field of view or the elapsed time is at least 12.5 seconds.

Within a given time series, the location of the visible edge of the sample at time zero is defined as the reference location from which the distance of propagation is measured for every image in that time series. The reference location is transcribed as a straight line onto each image in the time series. For a given image, the distance of propagation (L) is defined as the absolute distance between the transcribed reference location and the location of the water front in that image, when measured as a straight linear distance in the direction of propagation. For every image in a time series the position of the water front is visually determined, and the distance of propagation is measured and recorded. For every image in a time series, the elapsed time for that image is calculated and is recorded alongside the corresponding distance of propagation measured in that image. All measured distances are measured in micrometers.

During the wetting process, if the location of the bulk of the sample moves (e.g., floats and slides) relative to the reference line location then the images in that time series are unsuitable for providing accurate measurements and are discarded. Localized movement of some sample material due to dissolution is acceptable and does not require the time series to be discarded. Data can be measured from images in a time series wherein the propagating water front is approximately parallel to the edge of the sample visible in the field of view at time zero, and maintains that approximate orientation as the front advances. Data can also be measured from images wherein the water front is not completely straight and parallel to the visible edge of the web, in which case the location of the front is deemed to be a straight line parallel to the edge of the sample and located at approximately the average distance between the water front and the sample edge, as averaged across the length of the front visible within the field of view. Suitable video images from at least three replicate samples are required to be measured for each material being tested.

All measured distance values (L) are converted to meters. For each distance measurement, the elapsed time (t) in seconds is defined as the difference in time between the time of capture for the measured image and the time zero for that time series of images. The data from a time series are plotted to show Distance (L) in meters (as the y-axis ordinate) and elapsed Time (t) in seconds (as the x-axis abscissa). A curve is then fit to the plotted data using software such as SigmaPlot Version 11 (SYSTAT Software Inc., San Jose, Calif., U.S.A.) or equivalent. The curve fitted to the Distance versus Time data is a single, two-parameter exponential ‘Rise to Maximum’ curve as expressed by the following equation:

L=α(1−exp^{-βt})

Wherein:
α and β are the two curve-fitting parameters;
L is the linear distance of propagation travelled by the water front for a given time point since time zero, in meters; and
t is the elapsed time since time zero for a given time point, in seconds.

The Initial Water PropagationRate (ν(0)) is the intrinsic propagation rate prior to dissolution of the web and is defined as the time derivative of the curve fitted to the Distance versus Time data calculated for the time point t=0 using the following equation:

ν(0)=αβ

Wherein:

α and β are the two curve-fitting parameters;

The Initial Water PropagationRate (ν(0)) reported for a material being tested is the average value of (ν(0)) in meters per second, calculated as the average of the values determined from at least three replicate samples.

### Hydration Value Test Method

One of skill understands that obtaining a suitable sample from a fibrous article may involve several preparation steps, which may include the removal of lotions or fluids coating the article and/or fibrous element, and the separation of the various components from each other and from other components of the finished article. Furthermore, one of skill understands it is important to ensure that preparation steps for testing a fibrous element do not damage the sample to be tested or alter the characteristics to be measured. A clean fibrous element is the intended starting point for the measurement.

The Hydration Value of fibrous elements is determined from the testing of single fibrous elements. These single fibrous element tests are conducted using an upright compound light microscope, such as a Nikon Eclipse LV100POL (Nikon Instruments Inc., Melville, N.Y., U.S.A.) or equivalent. The microscope is equipped with long-working distance, flat-field corrected objective lenses of 10× or 20× magnification, such as Nikon CF Plan EPI ELWD (Nikon Instruments Inc., Melville, N.Y., U.S.A.) or equivalent. The microscope is also equipped with a high-speed video camera capable of capturing at least 200 frames s⁻¹ for 12.5 seconds, with at least 1024×512 pixels per frame, while capturing images having a minimum spatial resolution of 1.5 μm per pixel or higher resolution (i.e., a higher resolution corresponds to less distance per pixel). Suitable cameras include the Phantom V310 (Vision Research Inc., Wayne, N.J., U.S.A.) or equivalent. The microscope is aligned and spatial measurements in the x-y image plane are calibrated using a stage micrometer. Fibrous element samples are imaged and measured in brightfield transmission mode. Computer software programs may be used to control the video camera and to assist in the capture and spatial measurement analysis of images. Suitable software programs include Image-Pro Premier (64-bit, version 9.0.4, or equivalent) (Media Cybernetics Inc., Rockville, Md., U.S.A.).

Single fibrous element samples are prepared from a web by using fine-tip forceps or similar tools to extract single fibrous elements. An extracted fibrous element is suitable for analysis only if it is a single fibre or a composite bundle of approximately parallel fibrils, is unconnected to other fibrous elements, has a length that is at least 50 times than the element's average width, and neither end of the fibrous element is frayed or splayed. Fibrous elements may be gently teased apart from other fibrous elements via forceps, and may be trimmed at the ends using a new sharp razor blade. At all times care is taken not to flatten, kink, pinch nor damage the fibrous element. A suitable extracted fibrous element is placed lengthwise on a standard glass microscope slide with the fibrous element oriented with its length running parallel to the long axis of the slide. Taking care not to apply any additional pressure to the fibrous element, a glass microscope coverslip (thickness number 1.5) is gently lowered until it rests on top of the fibrous element. The slide-mounted fibrous element is placed onto the specimen stage of the microscope and its image is brought into focus under the 10× or 20× objective lens.

While capturing time-stamped photomicrograph video images of a mounted single fibrous element, laboratory grade filtered deionized (DI) water is slowly dispensed onto the slide using a 1 mL syringe filled with 23° C.±2° C. DI water. The water is dispensed at an edge of the coverslip which is perpendicular to the fibrous element's long axis. The water is dispensed such that it wicks under the coverslip until the water front gently touches one end of the fibrous element without causing the coverslip to float and slide away. While care is taken not to dislodge the fibrous element or coverslip, the water is dispensed quickly enough such that the air space under the coverslip is flooded with water within 5 seconds. The movement of the water front and its contact with the fibrous element is captured in the photomicrograph video images. The capture of video images is continued at least until the fibrous element is completely hydrated, in order to observe the swelling process of the fibrous element during hydration. After making initial contact at the fibrous element's end, the water front advances along the length of the fibrous element. Data is measured from video images wherein the advancing front of water is perpendicular to the fibrous element's long axis at the time of initial contact and maintains that orientation approximately evenly up both sides of the fibrous element as the front advances. A measurement location is unsuitable for providing accurate data if the advancing water front does not contact both sides of the fibrous element simultaneously at that measurement location. A measurement location is therefore discarded if the difference between the time points at which each side of the fibrous element comes into contact with water is a difference of more than 0.01 seconds at that location.

To determine the Hydration Value, linear spatial measurements across the diameter of the fibrous element are made from time series' of images extracted from captured videos. Each time series covers the timespan from just prior to the observation of water in the field of view through until when the fibrous element in the image is completely hydrated. Water penetrates into the fibrous element simultaneously from both sides inward toward the core, creating two fronts of hydration as the water penetrates. The positions of the hydration fronts inside the fibrous element are identified by visual observation of the captured images. Determination of the positions of the hydration fronts is facilitated by observing the change in opacity or whiteness which occurs when the material hydrates. Complete hydration at a given measurement location is defined as occurring when the opposing hydration fronts penetrating inside the fibrous element meet and thus the unhydrated core diameter at that location is zero.

From a captured video, the first frame in which the water front is observed is extracted and saved as the first frame of a time series. The time series is then extended by adding subsequent frames from the video that are temporally spaced apart approximately every 0.05 seconds. Additional images are extracted at the above temporal spacing and added to the time series, until the time series spans the period from with the first observation of water through to the complete hydration of the fibrous element.

At least two measurement locations are selected along the length of the long axis of the fibrous element within the first image in each time series of extracted images. The same two or more selected measurement locations are transcribed onto each subsequent image in that time series. Each selected measurement location is to be separated from adjacent measurement locations and from the physical end of the single fibrous element by a distance of at least ten times the average width of that single fibrous element. Locations are unsuitable for selection if the width of the fibrous element at that location differs from the average width of the element in that field of view by more than +/−30%. For each type of fibrous element, at least six locations in total are measured, located on at least three replicate single fibrous element samples. Each measurement location has its own independent time zero, which is defined as the time of capture associated with the image frame in which an hydration front is first visible inside the fiber at that measurement location. For a measurement location in a given image, the elapsed time (t) in seconds is defined as the difference in time between the time of capture for the given image and the time zero for that measurement location.

Within each time series of images, two different diameters are measured at each selected measurement location. All measured diameters are measured in micrometers. The first diameter measured is the initial diameter (termed “initial diameter”) of the dry fibrous element prior to its contact with water. This initial diameter is measured only once for any given location in any given time series and that measurement are made in the first image of the time series. The second diameter measured (termed “unhydrated core diameter”) is the diameter of the unhydrated core located between the hydration fronts penetrating into the fibrous element at a given time point after contact with water. This unhydrated core diameter is measured in every image of the time series after time zero. The unhydrated core diameter is defined by the location of the water fronts penetrating into the fibrous element from the side edges of the element. Complete hydration is defined as when the opposing penetrating hydration fronts meet inside the fibrous element and thus the unhydrated core diameter is zero.

The following equation is used to calculate a Hydration Value (h) for each measurement location in each image of a time series after time zero:

$$h = \frac{( initial diameter ) - ( unhydrated core diameter )}{2}$$

Where, at a given measurement location within a given image from a time series:
Unhydrated Core Diameter=the diameter of the unhydrated core located between the penetrating hydration fronts within the fibrous element;
Initial Diameter=the diameter of that same fibrous element at that same measurement location prior to contact with water.

For each selected measurement location within a time series after time zero, all calculated Hydration Values (h) are converted to meters and plotted (as the y-axis ordinate) versus the square root of the elapsed time (t) in seconds (as the x-axis abscissa). A single Hydration Value in m/s^{1/2} is then calculated for each measurement location, and is defined as the slope of the straight line resulting from a simple linear regression analysis (least squares) of the plotted data. The Hydration Value reported for each type of fibrous element is the average of the Hydration Values determined from measurement locations on at least three replicate samples of that type of fibrous element.

### Swelling Value Test Method

One of skill understands that obtaining a suitable sample from a fibrous article may involve several preparation steps, which may include the removal of lotions or fluids coating the article and/or fibrous element, and the separation of the various components from each other and from other components of the finished article. Furthermore, one of skill understands it is important to ensure that preparation steps for testing a fibrous element do not damage the sample to be tested or alter the characteristics to be measured. A clean fibrous element is the intended starting point for the measurement.

The Swelling Value of fibrous elements is determined from the testing of single fibrous elements. These single fibrous element tests are conducted using an upright compound light microscope, such as a Nikon Eclipse LV100POL (Nikon Instruments Inc., Melville, N.Y., U.S.A.) or equivalent. The microscope is equipped with long-working distance, flat-field corrected objective lenses of 10× or 20× magnification, such as Nikon CF Plan EPI ELWD (Nikon Instruments Inc., Melville, N.Y., U.S.A.) or equivalent. The microscope is also equipped with a high-speed video camera capable of capturing at least 200 frames s⁻¹ for 12.5 seconds, with at least 1024×512 pixels per frame, while capturing images having a minimum spatial resolution of 1.5 μm per pixel or higher resolution (i.e., a higher resolution corresponds to less distance per pixel). Suitable cameras include the Phantom V310 (Vision Research Inc., Wayne, N.J., U.S.A.) or equivalent. The microscope is aligned for Koehler Illumination and spatial measurements in the x-y image plane are calibrated using a stage micrometer. Fibrous element samples are imaged and measured in brightfield transmission illumination mode. Computer software programs may be used to control the video camera and to assist in the capture and spatial measurement analysis of images. Suitable software programs include Image-Pro Premier 64-bit, version 9.0.4 (Media Cybernetics Inc., Rockville, Md., U.S.A.), or equivalent.

Single fibrous element samples are prepared from a web by using fine-tip forceps or similar tools to extract single fibrous elements from the web. Fibrous elements may be gently teased apart from other fibrous elements via forceps, and may be trimmed at the ends using a new sharp razor blade. An extracted fibrous element is suitable for analysis only if it is a single fibre or a composite bundle of approximately parallel fibrils, is unconnected to other fibrous elements, has a length that is at least 50 times than the element's average width, and neither end of the fibrous element is frayed or splayed. At all times care is taken not to flatten, kink, pinch nor damage the fibrous element. A suitable extracted fibrous element is placed lengthwise on a standard glass microscope slide with the fibrous element oriented with its length running parallel to the long axis of the slide. Taking care not to apply any additional pressure to the fibrous element, a glass microscope coverslip (thickness number 1.5) is gently lowered until it rests on top of the fibrous element. The slide-mounted fibrous element is placed onto the specimen stage of the microscope and its image is brought into focus under the 10× or 20× objective lens.

While capturing time-stamped photomicrograph video images of a mounted single fibrous element, laboratory grade filtered deionized (DI) water is slowly dispensed onto the slide using a 1 mL syringe filled with 23° C.±2° C. DI water. The water is dispensed at one edge of the coverslip which is perpendicular to the fibrous element's long axis. The water is dispensed such that it wicks under the coverslip and the water front gently touches one end of the fibrous element without causing the coverslip to float or slide away. While care is taken not to dislodge the fibrous element or the coverslip, the water is dispensed quickly enough such that the air space under the coverslip is flooded with water within 5 seconds. The movement of the water front and its contact with the fibrous element is captured in the photomicrograph video images. The capture of video images is continued at least until the fibrous element is completely hydrated, in order to observe the swelling process of the fibrous element during hydration. After making initial contact at the fibrous element's end, the water front advances along the length of the fibrous element. Data is measured from video images wherein the advancing front of water is perpendicular to the fibrous element's long axis at the time of initial contact and maintains that orientation approximately evenly up both sides of the fibrous element as the water front advances. A measurement location is unsuitable for providing accurate data if the advancing water front does not contact both sides of the fibrous element simultaneously at that measurement location. A measurement location is therefore discarded if the difference between the time points at which each side of the fibrous element comes into contact with water is a difference of more than 0.01 seconds at that location.

To determine the Swelling Value, linear spatial measurements along the diameter of the fibrous element are made from time series' of images extracted from captured videos. Each time series covers the timespan from just prior to the observation of water in the field of view through until when the fibrous element in the image is completely hydrated. Water penetrates into the fibrous element simultaneously from both sides inward toward the core, creating two fronts of hydration as the water penetrates. The positions of the hydration fronts inside the fibrous element are identified by visual observation of the captured images. Determination of the positions of the hydration fronts is facilitated by observing the change in opacity or whiteness which occurs when the material hydrates. Complete hydration at a given measurement location is defined as occurring when the opposing hydration fronts penetrating inside the fibrous element meet and thus the unhydrated core diameter at that location is zero.

From a captured video, the first frame in which the water front is observed is extracted and saved as the first frame of a time series. The time series is then extended by adding subsequent frames from the video that are temporally spaced apart approximately every 0.05 seconds. Additional images are extracted at the above temporal spacing and added to the time series, until the time series spans the period from with the first observation of water through to the complete hydration of the fibrous element.

At least two measurement locations are selected along the length of the long axis of the fibrous element within the first image in each time series of extracted images. The same two or more measurement locations selected are transcribed onto each subsequent image in that time series. Each selected measurement location is to be separated from adjacent measurement locations and from the physical end of the single fibrous element by a distance of at least ten times the average width of that single fibrous element. Locations are unsuitable for selection if the width of the fibrous element at that location differs from the average width of the element in that field of view by more than +/−30%. For each type of fibrous element, at least six locations in total are measured, located on at least three replicate single fibrous element samples. The time point at which the advancing water front first contacts the edges of the fibrous element at the measurement location is considered to be the time zero for that measurement location.

Within each time series of images, three different diameters are measured at each selected measurement location. All measured diameters are measured in micrometers. Two of these diameters are remeasured repeatedly in different images of the time series (i.e., at different time points). The first diameter measured is the initial diameter (termed “initial diameter”) of the dry fibrous element prior to its contact with water. This initial diameter is measured only once for any given location in any given time series, and that measurement is made in the first image of the time series.

The second diameter measured (termed “wet diameter”) is the diameter of the fibrous element at a given time point after contact with water. This wet diameter is measured in every image of the time series after time zero (i.e., in every image after the time point at which water contacted the measurement location).

The third diameter measured (termed “unhydrated core diameter”) is the diameter of the unhydrated core located between the hydration fronts penetrating into the fibrous element at a given time point after contact with water. This unhydrated core diameter is measured in every image of the time series after time zero (i.e. every image after the time point at which water contacted the measurement location). The unhydrated core diameter is defined by the location of the hydration fronts penetrating into the fibrous element from both side edges of the element. Determination of the positions of the hydration fronts is facilitated by visually observing the change in opacity or whiteness of the fibrous material which occurs as the material hydrates. Complete hydration is defined as when the opposing penetrating hydration fronts meet inside the fibrous element and thus the unhydrated core diameter is zero.

The following equation is used to calculate a Swelling Value (s) for each measurement location in each image of a time series after time zero:

$$s = \frac{{( Wet Diameter )}^{2} - {( Unhydrated Core Diameter )}^{2}}{{( Initial Diameter )}^{2} - {( Unhydrated Core Diameter )}^{2}}$$

Where, at a given measurement location within a given image from a time series:
Wet Diameter=the diameter of the fibrous element after contact with water;
Unhydrated Core Diameter=the diameter of the unhydrated core located between the penetrating hydration fronts within the fibrous element;
Initial Diameter=the diameter of that same fibrous element at that same measurement location prior to contact with water.

The Swelling Value (S) reported for each type of fibrous element is the average of all Swelling Values (s) calculated from all replicate samples, measurement locations, and time series, of that type of fibrous element.

### Viscosity Value Test Method

Two to 3 grams of the sample material to be tested is weighed out into a mixing jar (borosilicate glass with screw cap of about 30 mm diameter, about 60 mm height, volume of about 15 mL and plastic screw cap lid).

When the sample material is a pre-formed web or other dry form of material, sufficient laboratory-grade, filtered, deionized water (DI water), is weighed out into the mixing jar with the sample, such that the mass of the water equals three times the mass of the web or dry form sample (i.e., to give a final concentration of water of 75% (wt/wt)).

When the sample material is a liquid premix or other wet form of material, sufficient DI water is weighed out into the mixing jar such that the resultant aqueous solution has a final concentration of water of 75% (wt/wt). A wet form sample that has a water content which is already greater than 75% (wt/wt) is first air-dried in a vacuum desiccator until the water concentration falls below 75%, and is subsequently adjusted with sufficient DI water to result in a final concentration of water of 75% (wt/wt). Water concentrations may be determined via Karl Fischer Titration instruments.

To thoroughly mix and dissolve the sample material into solution, a stir bar is placed into the mixing jar containing the sample and water, and the jar sealed with its lid then mounted onto an orbital shaker mixing device, such as the VWR Model 3500, Catalog no. 89032-092 (VWR, Radnor, Pa., U.S.A.). The jar and solution therein is then shaken for 24 hours at a speed setting which delivers approximately 85 revolutions/min. After 24 hours, the sample is visually checked to determine if it is well mixed as indicated by the absence of any large unmixed chunks, or residual materials along the neck of the jar. Well mixed sample solutions are then tested to determine the Viscosity Value. Sample solutions that are not yet well mixed are returned to the mixing device and shaken for another 24 hours of shaking.

For a given well mixed sample prepared as above, the viscosity reported is the Viscosity Value as measured by the following method, which generally represents the zero-shear viscosity (or zero-rate viscosity). Viscosity measurements are made with a TA Discovery HR-2 Hybrid Rheometer (TA Instruments, New Castle, Del., U.S.A.), and accompanying TRIOS software version 3.0.2.3156. The instrument is outfitted with a 40 mm stainless steel parallel plate (TA Instruments catalog no. 511400.901) and Peltier plate (TA Instruments catalog no. 533230.901). The calibration is done in accordance with manufacturer recommendations. A refrigerated, circulating water bath set to 25° C. is attached to the Peltier plate.

Measurements are made on the instrument with the following procedures and settings selected: Conditioning Step (pre-condition the sample) under “Settings” label, initial temperature: 25° C., pre-shear at 5.0 s⁻¹ for 1 minute, equilibrate for 2 minutes; Flow-Step (measure viscosity) under “Test” Label, Test Type: “Steady State Flow”, Ramp: “shear rate 1/s” from 0.001 s⁻¹ and 1000 s⁻¹, Mode: “Log”, Points per Decade: 15, Temperate: 25° C., Percentage Tolerance: 5, Consecutive with Tolerance: 3, Maximum Point Time: 45 s, Gap set to 500 micrometers, Stress-Sweep Step is not checked; Post-Experiment Step under “Settings” label; Set temperature: 25° C.

More than 1.25 mL of the well mixed test sample solution to be measured is dispensed through a pipette onto the center of the Peltier plate. The 40 mm plate is slowly lowered to 550 micrometers, and the excess sample is trimmed away from the edge of the plate with a rubber policeman trimming tool or equivalent. The plate is then lowered to 500 micrometers (gap setting) prior to collection of the data.

Data points which were collected with an applied rotor torque of less than 1 micro-N·m (i.e., less than ten-fold the minimum torque specification) are discarded. Data points which possess a measured strain of less than 3 are also discarded. The remaining data points are used to create a plot of the measured Viscosity Values versus shear rate, on a log-log scale. These plotted data points are analyzed in one of three ways to determine the Viscosity Value of the sample solution, as given below:

First, if the plot indicates that the sample is Newtonian, in that all Viscosity Values fall on a plateau within +/−20% of the Viscosity Value measured closest to 1 micro-N·m, then the viscosity is determined by selecting the “Analysis” tab, selecting the “Newtonian” option, pushing the “Match” button, selecting the limits in accordance with the torque and strain specifications given above and hitting “Start”.

Second, if the plot reveals a plateau in which the Viscosity Values do not vary by at least +/−20% at low shear rates, and reveals a sharp nearly-linear decrease in Viscosity Values in excess of the +/−20% at higher shear rates, then the viscosity is determined by selecting the “Analysis” tab, selecting the “Best Fit Flow (Viscosity vs. Rate)” option, selecting the limits in accordance with the torque and strain specifications given above and hitting “Start”.

Third, if the plot indicates that the sample is only shear-thinning, in that there is only a sharp, nearly-linear decrease in Viscosity Values, then the material is characterized by a Viscosity Value which is taken as the largest viscosity in the plotted data, generally this will be a Viscosity Value measured close to 1 micro-N·m of applied torque.

The Viscosity Value reported is the average value of the replicate samples prepared, expressed in units of Pa·s.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A soluble fibrous structure comprising a plurality of fibrous elements comprising one or more fibrous element-forming materials and one or more active agents that are releasable from the fibrous elements when exposed to conditions of intended use, wherein the soluble fibrous structure exhibits one or more of the following properties:
a. the soluble fibrous structure exhibits an Initial Water Propagation Rate of greater than about 5.0×10⁻⁴ m/s as measured according to the Initial Water Propagation Rate Test Method;
b. at least one fibrous element within the soluble fibrous structure exhibits a Hydration Value of greater than about 7.75×10⁻⁵ m/s^{1/2} as measured according to the Hydration Value Test Method;
c. at least one fibrous element within the soluble fibrous structure exhibits a Swelling Value of less than about 2.05 as measured according to the Swelling Value Test Method;
d. at least one fibrous element within the soluble fibrous structure comprises a fibrous element-forming composition that exhibits a Viscosity Value of less than about 100 Pa·s as measured according to the Viscosity Value Test Method;
e. at least one fibrous element within the soluble fibrous structure exhibits a Viscosity Value of less than about 100 Pa·s as measured according to the Viscosity Value Test Method; and
f. the soluble fibrous structure exhibits a Viscosity Value of less than about 100 Pa·s as measured according to the Viscosity Value Test Method.

2. The soluble fibrous structure according to claim 1 wherein one or more of the fibrous elements are water-soluble.

3. The soluble fibrous structure according to claim 1 wherein the fibrous elements comprise one or more filaments.

4. The soluble fibrous structure according to claim 1 wherein at least one of the one or more active agents comprises a surfactant.

5. The soluble fibrous structure according to claim 4 wherein the surfactant is selected from the group consisting of: anionic surfactants, cationic surfactants, nonionic surfactants, zwitterionic surfactants, amphoteric surfactants, and mixtures thereof.

6. The fibrous structure according to claim 1 wherein the one or more active agents is selected from the group consisting of: fabric care active agents, dishwashing active agents, carpet care active agents, surface care active agents, air care active agents, and mixtures thereof.

7. The soluble fibrous structure according to claim 1 wherein at least one of the one or more active agents is in the form of a particle exhibiting a median particle size of 20 μm or less as measured according to the Median Particle Size Test Method.

8. The soluble fibrous structure according to claim 7 wherein the particle comprises a perfume microcapsule.

9. The soluble fibrous structure according to claim 1 wherein the soluble fibrous structure comprises one or more particles.

10. The soluble fibrous structure according to claim 9 wherein at least one of the particles is present within at least one of the fibrous elements.

11. The soluble fibrous structure according to claim 9 wherein at least one of the particles is within the soluble fibrous structure inter-fibrous elements.

12. The soluble fibrous structure according to claim 1 wherein the one or more fibrous element-forming materials comprises a polymer.

13. The soluble fibrous structure according to claim 12 wherein the polymer is selected from the group consisting of: pullulan, hydroxypropylmethyl cellulose, hydroxyethyl cellulose, hydroxypropyl cellulose, polyvinyl pyrrolidone, carboxymethyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, carboxylated polyvinyl alcohol, sulfonated polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, and mixtures thereof.

14. The soluble fibrous structure according to claim 1 wherein the fibrous structure exhibits a basis weight of from about 1 g/m² to about 10000 g/m².

15. The soluble fibrous structure according to claim 1 wherein the fibrous elements are present in the fibrous structure in two or more layers.

16. The soluble fibrous structure according to claim 1 wherein at least one of the fibrous elements exhibits an average diameter of less than 50 μm as measured according to the Diameter Test Method.

17. The soluble fibrous structure according to claim 1 wherein the fibrous structure exhibits a dissolution time of 600 seconds or less as measured according to the Dissolution Test Method.

18. The soluble fibrous structure according to claim 1 wherein at least one of the fibrous elements comprises a coating composition present on an external surface of the fibrous element.

19. The soluble fibrous structure according to claim 1 wherein the soluble fibrous structure exhibits an Initial Water Propagation Rate of greater than about 7.75×10⁻⁴ m/s as measured according to the Initial Water Propagation Rate Test Method.

20. The soluble fibrous structure according to claim 1 wherein the soluble fibrous structure comprises at least one fibrous element that exhibits a Hydration Value of greater than about 9.0×10⁻⁵ m/s^{1/2} as measured according to the Hydration Value Test Method.

21. The soluble fibrous structure according to claim 1 wherein the soluble fibrous structure comprises at least one fibrous element that exhibits a Swelling Value of less than about 2.0 as measured according to the Swelling Value Test Method.

22. The soluble fibrous structure according to claim 1 wherein the soluble fibrous structure comprises at least one fibrous element comprising a fibrous element-forming composition that exhibits a Viscosity Value of less than about 80 Pa·s as measured according to the Viscosity Value Test Method.

23. The soluble fibrous structure according to claim 1 wherein the soluble fibrous structure comprises at least one fibrous element comprising a fibrous element-forming composition such that the fibrous element exhibits a Viscosity Value of less than about 80 Pa·s as measured according to the Viscosity Value Test Method.

24. The soluble fibrous structure according to claim 1 wherein the soluble fibrous structure comprises at least one fibrous element comprising a fibrous element-forming composition such that the soluble fibrous structure exhibits a Viscosity Value of less than about 80 Pa·s as measured according to the Viscosity Value Test Method.

25. The soluble fibrous structure according to claim 1 wherein the soluble fibrous structure exhibits a GM Tensile Strength of greater than of about 200 g/in as measured according to the Tensile Test Method.

26. The soluble fibrous structure according to claim 1 wherein the soluble fibrous structure exhibits a GM Peak Elongation of less than about 1000% as measured according to the Tensile Test Method.

27. A multi-ply fibrous structure comprising at least one ply of a soluble fibrous structure according to claim 1.
